(19) United States
(12) Patent Application Publication
(10) Pub. No.: US 2021/0170056 A1
(43) Pub. Date: Jun. 10, 2021

(54) SLIPPERY MICROPROPELLERS PENETRATE THE VITREOUS HUMOR

(71) Applicant: Max-Planck-Innovation GMBH, Munich (DE)

(72) Inventors: Zhiguang Wu, Stuttgart (DE); Tian Qiu, Stuttgart (DE); Peer Fischer, Freiburg (DE)

(21) Appl. No.: 16/641,059

(22) PCT Filed: Aug. 21, 2018
(86) PCT No.: PCT/EP2018/072501

§ 371 Date: Feb. 21, 2020

(51) Int. Cl. A61K 49/18; A61K 9/00; A61K 45/06; A61K 41/00

(52) U.S. Cl. CPC A61K 49/1824; A61K 9/0009; B82Y 5/00; A61K 41/00; A61K 45/06

(57) ABSTRACT

Microparticles actively propel through the vitreous humour and reach the retina in porcine eyes. The slippery micro helical propellers are constructed by the combination of glancing angle deposition technique and the fusion of the slippery liquid layer. The magnetically propulsion in the vitreous humour relies on the matched size of the propeller to the collagen network of the vitreous, and the anti-adhesion coating of the collagen fiber bundles. Clinical optical coherence tomography observed the displacement of the slippery micropropellers through the vitreous to the macular area on the retina. The slippery micropropellers realize the controllable massive movements to the retina in 30 mins, while exerting the travelling distance of above one centimeter. The injection of the slippery micropropellers, the magnetically-powered controllable propulsion in the vitreous, and the optical coherence tomography imaging technique, constitute an intact method for rapid targeted ocular delivery, providing a promising approach towards ophthalmologic applications.

### CROSS-REFERENCE TO RELATED APPLICATIONS

This application is a national stage application of and claims priority to and the benefit of co-pending PCT patent application PCT/EP2018/072501, entitled Slippery Micropropellers Penetrate the Vitreous Humor, filed Aug. 21, 2018, which is incorporated herein by reference in its entirety. The PCT application claims priority to EPO Patent Application No. 17187924.0. This application also claims priority to and the benefit of EPO Patent Application No. 17187924.0, which is also incorporated herein by reference in its entirety.

### FIELD OF THE INVENTION

The present invention concerns a method which facilitates the diffusion or active transport of a particle through a medium. The present invention moreover concerns a new particle.

### BACKGROUND OF THE INVENTION

Intravitreal delivery of the therapeutic and imaging nanoparticles promised considerable potential applications in the field of the ocular medicine, while the slow and random passive diffusion of the particles in vitreous are prompting novel strategies for rapid delivery to target site in the back of the eye.

Ocular drug delivery plays an important role in ophthalmology as it treats diverse diseases such as diabetic retinopathy, glaucoma, and diabetic macular edema.[[¹]] See Patel, A., Cholkar, K., Agrahari, V. & Mitra, A. K. Ocular drug delivery systems: An overview. World journal of pharmacology 2, 47-64, doi:10.5497/wjp.v2.i2.47 (2013). Although topical administration is currently available to treat diseases in the anterior eye segment including cornea, ciliary body and lens, [[²]] see Resnikoff, S. et al. Global data on visual impairment in the year 2002. Bulletin of the world health organization 82, 844-851 (2004), the delivery to the posterior part of the eye via topical or systematic administration is very ineffective and difficult due to the drug loss from the ocular surface, lacrimal fluid-eye barrier, and retina-blood barrier.[[^{3,4}]] See Hughes, P. M., Olejnik, O., Chang-Lin, J.-E. & Wilson, C. G. Topical and systemic drug delivery to the posterior segments. Advanced drug delivery reviews 57, 2010-2032 (2005); Urtti, A. Challenges and obstacles of ocular pharmacokinetics and drug delivery. Advanced drug delivery reviews 58, 1131-1135 (2006). To solve the issue, a variety of biomedical nanoparticles have been investigated for intravitreal injection and passive diffusion towards the retina.[[⁵⁻⁷]] See Zhou, H.-Y., Hao, J.-L., Wang, S., Zheng, Y. & Zhang, W.-S. Nanoparticles in the ocular drug delivery. International Journal of Ophthalmology 6, 390-396, doi:10.3980/j.issn.2222-3959.2013.03.25 (2013); Xu, Q. et al. Nanoparticle diffusion in, and microrheology of, the bovine vitreous ex vivo. Journal of controlled release 167, 76-84 (2013); Kasdorf, Benjamin T., Arends, F. & Lieleg, O. Diffusion Regulation in the Vitreous Humor. Biophysical Journal 109, 2171-2181, doi:https://doi.org/10.1016/j.bpj.2015.10.002 (2015). Passive diffusion suffers from long period of diffusion time and decreased activity of biomedical agents,[[⁸]] see Witkin, A. J. & Brown, G. C. Update on nonsurgical therapy for diabetic macular edema. Current opinion in ophthalmology 22, 185-189 (2011), moreover, it is a systematic approach without the preference of the target sites and increases the risk of side effects. [[⁹]] See Janoria, K. G., Gunda, S., Boddu, S. H. & Mitra, A. K. Novel approaches to retinal drug delivery. Expert opinion on drug delivery 4, 371-388 (2007). Therefore, it still remains challenging to rapidly and targeted deliver drugs through intravitreal administration.

In contrast to the conventional passive diffusion of the biomedical particles in the vitreous, active micro/nanopropellers provide a novel pathway for targeted drug delivery in the human body[[¹⁰⁻¹³]]. See Wang, J. (John Wiley & Sons, 2013); Qin, W. et al. Catalysis-Driven Self-Thermophoresis of Janus Plasmonic Nanomotors. Angewandte Chemie International Edition 56, 515-518 (2017); Fan, D. et al. Subcellular-resolution delivery of a cytokine through precisely manipulated nanowires. Nat Nano 5, 545-551, doi:http://www.nature.com/nnano/journal/v5/n7/abs/nnano.2010.104.html#suppl ementary-information (2010); Dai, B. et al. Programmable artificial phototactic microswimmer. Nature nanotechnology 11, 1087-1092 (2016). Learning from nature, biomimetic micro/nanopropellers demonstrated self-powered locomotion in various fluids by converting diverse energies into mechanical movement.^{[[14,15}]] See Katuri, J., Ma, X., Stanton, M. M. & Sanchez, S. Designing micro- and nanoswimmers for specific applications. Accounts of chemical research 50, 2-11 (2016); Lin, X., Wu, Z., Wu, Y., Xuan, M. & He, Q. Self-Propelled Micro-/Nanomotors Based on Controlled Assembled Architectures. Advanced Materials 28, 1060-1072, doi:10.1002/adma.201502583 (2016). Since the investigation for the propulsion of the acid-powered microrockets in rat stomach and the magnetic swarm movement of microrobotic flagella, [[^{16,17}]] see Gao, W. et al. Artificial micromotors in the mouse's stomach: A step toward in vivo use of synthetic motors. ACS nano 9, 117-123 (2015); Servant, A., Qiu, F., Mazza, M., Kostarelos, K. & Nelson, B. J. Controlled In Vivo Swimming of a Swarm of Bacteria-Like Microrobotic Flagella. Advanced Materials 27, 2981-2988, doi:10.1002/adma.201404444 (2015),which represents the first steps of the synthetic motor towards in vivo conditions, [[¹⁸]] see Peng, F., Tu, Y. & Wilson, D. A. Micro/nanomotors towards in vivo application: cell, tissue and biofluid. Chemical Society Reviews (2017), the pursuit of the propulsion in vivo has recently led to a number of synthetic propellers towards clinical biomedicine.[[¹⁹]] See Li, J., de Avila, B. E.-F., Gao, W., Zhang, L. & Wang, J. Micro/nanorobots for biomedicine: Delivery, surgery, sensing, and detoxification. Science Robotics 2, eaam6431 (2017). Among various propellers, the magnetically-powered propellers, which mimics the movement behavior of the bacteria flagella, demonstrate effective propulsion with precise velocity and direction control and without the need of external fuels or high-cost instruments. [[²⁰⁻²⁵]] See Xu, T., Gao, W., Xu, L. P., Zhang, X. & Wang, S. Micro-/Nanomachines: Fuel-Free Synthetic Micro-/Nanomachines (Adv. Mater. September 2017). Advanced Materials 29 (2017); Tottori, S. et al. Magnetic Helical Micromachines: Fabrication, Controlled Swimming, and Cargo Transport. Advanced Materials 24, 811-816 (2012); Zhang, L. et al. Anomalous coiling of SiGe/Si and SiGe/Si/Cr helical nanobelts. Nano letters 6, 1311-1317 (2006); Li, T. et al. Highly efficient freestyle magnetic nanoswimmer. Nano Letters (2017); 24 Ghosh, A. & Fischer, P. Controlled Propulsion of Artificial Magnetic Nanostructured Propellers. Nano Letters 9, 2243-2245, doi:10.1021/nl900186w (2009); Schamel, D. et al. Nanopropellers and Their Actuation in Complex Viscoelastic Media. ACS Nano 8, 8794-8801, doi:10.1021/nn502360t (2014).

Substantial efforts have been devoted to achieve the movement of micro/nanopropellers in real biological tissues, such as in the ocular vitreous. For example, Nelson's team reported the 200 μm translational movement of a beyond 200 μm-diameter cylindrical microrobot upon the external magnetic field. [[²⁶]] See Ullrich, F. et al. Mobility experiments with microrobots for minimally invasive intraocular Surgery Microrobot experiments for intraocular surgery. Investigative ophthalmology & visual science 54, 2853-2863 (2013). However, the long distance displacement of micropropellers has not yet been realized in the vitreous due to the strong obstruction of the collagen fibrils network. Active microrheology studies showed that the mesh size of the porcine vitreous is ˜500 nm, suggesting that nanoparticles with size well below this threshold are able to move unhindered. [[²⁷]] See Qiu, T., Schamel, D., Mark, A. G. & Fischer, P. in 2014 IEEE International Conference on Robotics and Automation (ICRA). 3801-3806. Moreover, we reported that magnetic helical nanopropellers of 120 nm in diameter and 400 nm in length are able to propel in porous hyaluronan solution, a model fluid mimicking the vitreous. [[²⁵]] See Schamel, D. et al. Nanopropellers and Their Actuation in Complex Viscoelastic Media. ACS Nano 8, 8794-8801, doi:10.1021/nn502360t (2014). These results indicate that if the size of the propellers is much smaller than the mesh size of the complex network, their interaction with the network is minimized, therefore, the propulsion of nanopropellers, but not micropropellers, is possible in the nanoporous medium. However, both the propulsive speed and load capacity of nanopropellers are limited due to their small size. Therefore, it is beneficial to make the propeller size as large as possible, for example to micrometer size, while it can still penetrate the porous biological media.

Slippery surfaces are known from the U.S. Pat. No. 9,121,306 B2 and from the paper by the same authors of the patent: Wong, T.-S. et al. Bioinspired self-repairing slippery surfaces with pressure-stable omniphobicity. A method for targeted delivery of therapeutic agents within the eye is known from the patent application publication document US 2004/0086572 A1.

### Problem According to the Invention

The present invention addresses the problem of providing a new method which facilitates the diffusion or active transport of a particle through a medium. The present invention moreover addresses the problem of providing a new particle.

### Solution According to the Invention and Preferred Embodiments

The problem is solved by providing a method according to claim 1. It is also solved by providing a particle pursuant to claim 2. Preferably, the particle is coated with at least one solid layer linked to the surface of the particle and at least one liquid layer that surrounds the solid layer. Other preferred embodiments of the invention are provided in the dependent claims and in the following description. Preferred features of the invention may be applied alone or in combination are discussed in the dependent claims, description below and the figures. For the realisation of the invention in its various embodiments, the features disclosed in the present description, claims and drawings can be of relevance individually as well as in any combination.

Preferably, the characteristic size of the particle is equal or smaller than the mesh size of the medium.

Preferably, the characteristic size of the particle is larger than the mesh size of the medium, preferably not larger than 1 000 times, more preferably not larger than 10 times of the mesh size, and/or characterized in that the solid layer has a thickness of between 0.2 nm and 20 μm and/or the liquid layer has a thickness between 0.5 nm and 500 μm.

Preferably, the coating material of the solid layer or the liquid layer contain one or more components from the group of components comprising: poly(ethylene oxide), poly(4-styrenesulfonic acid), poly(sodium 4-styrenesulfonate), polyethylene glycol, siloxane, perfluorocarbon, negative charged polyelectrolytes, hyaluronic acid, poloxamer, enzymes, albumin, polysaccharides, poly(vinyl acetate), poly(vinylpyrrolidone).

Preferably, the particle is dispersed in aqueous solution, prior to the application in the medium and/or characterized in that particles are directly applied in the medium.

At least one stabilizer is added in the aqueous solution to keep the particles dispersed. The stabilizer includes at least one component from poly(vinyl alcohol), polyvinylpyrrolidone, poly(ethylene oxide), polyethylene glycol, poly(4-styrenesulfonic acid), poly(sodium 4-styrenesulfonate), hyaluronic acid, poloxamer, starch, dextrin, chitosan, alginate, isolated soy protein, gelatin, catalase, whey protein, albumin, histones, carrageenan, xanthan gum, phenylpropanamide, sodium benzenesulfonate.

Preferably, the particle has therapeutic functions, and/or the particle is used to aid biomedical imaging or diagnostics imaging, and/or the particle is radioactive, or generate heat or light radiation under an external stimulus, and/or the particle is associated, or contacts a therapeutic agent.

Preferably, the particle has a chiral or modified chiral part, and/or the particle has a part in helical shape, and/or the particle has a magnetic moment.

Preferably, the particle diffuses through a medium that is biologically relevant, including human or animal vitreous humor, mucus, synovial fluids, lymphatic fluids, cells, connective tissues, the tissues of brain, nerve, heart, lung, kidney, blood vessel, liver, pancreas, gall bladder, GI tract, urinary tract, testicle, penis, female reproductive tract, breast, prostate, ear, nose, appendix, joint and bone, or the particle is transported by the application of an external force or torque through a medium that is biologically relevant, including human or animal vitreous humor, mucus, synovial fluids, lymphatic fluids, cells, connective tissues, the tissues of brain, nerve, heart, lung, kidney, blood vessel, liver, pancreas, gall bladder, GI tract, urinary tract, testicle, penis, female reproductive tract, breast, prostate, ear, nose, appendix, joint and bone.

Preferably, the motion of the particle is induced remotely by means of a magnetic field.

A preferred method for producing a particle with a coating is characterized in that the method comprises the steps of:
fabrication of the particle in defined shape
coating a solid layer that links to the surface of the particles
coating a liquid layer that fuses with the said solid layer

A preferred method for utilizing a particle with a coating is characterized in that the method comprises the steps of:
suspend the particle in an aqueous solution
injection the suspension into a medium
apply a magnetic field to induce the movement of the particle
observe the movement with an imaging technique A preferred method for utilizing a particle with a coating is characterized in that the method comprises the steps of:
disperse the particle into a medium
apply a magnetic field to induce the movement of the particle
observe the movement with an imaging technique

Preferably, the magnetic field is altered based on the feedback of the imaging results, and the particles are guided to a target location in the said medium.

In the context of the present invention, the “characteristic size” of a particle is the maximal length on any cross-section, which is perpendicular to the moving direction of the particle and intersects with the particle.

In the context of the present invention, the “mesh size” of the medium is the average pore size of a porous medium.

The terms “particle” and “microparticle” are used synonymously in this text. The prefix “micro” is merely meant to indicate that in some applications the particle may be small in some sense or another, for example as compared to the volume in which it is moving. The preferred particle is a propeller. The terms “propeller” and “micropropeller” are used synonymously in this text. The prefix “micro” is merely meant to indicate that in some applications the propeller may be small in some sense or another, for example as compared to the volume in which it is moving.

The preferred particle is a helix. The terms “helix” and “microhelix” are used synonymously in this text. The prefix “micro” is merely meant to indicate that in some applications the propeller may be small in some sense or another, for example as compared to the volume in which it is moving.

In the context of the present invention, “slippery” means that the particle is provided with at least one coating that facilitates locomotion, in particular to avoid adhesion to the medium as further detailed below.

The earlier invention “Propeller and method in which a propeller is set into motion,” which has been submitted as yet unpublished European patent application 17166356 on 12 Apr. 2017, is part of the present invention, and accordingly, the description of the latter application has been included into the present description (starting below the heading “Propeller and method in which a propeller is set into motion”) and the figures of the latter application have been included into the present figures in full. This means that ia the definitions provided below the heading “Propeller and method in which a propeller is set into motion” shall apply to the entire present application. Moreover, the present invention encompasses any combination of features disclosed before the heading “Propeller and method in which a propeller is set into motion” with features disclosed after the heading “Propeller and method in which a propeller is set into motion”. The same applies, mutatis mutandis to the figures and the claims and to combinations of any part of the written description, any of the figures and any of the claims.

In particular, in some embodiments of the present invention, the propeller according to the invention is set into locomotion by means of a method as described in the text following the heading “Propeller and method in which a propeller is set into motion”. Also, in some embodiments of the invention, the propeller according to the invention is a propeller s described in the text following the heading “Propeller and method in which a propeller is set into motion”. Moreover, in some embodiments of the invention the propeller according to the invention is manufactured by one of the methods described in the text following the heading “Propeller and method in which a propeller is set into motion”.

Here, we report, ia, the first microparticles that actively propel through the vitreous humour and reach the retina in porcine eyes. A preferred microparticle is a slippery micropropeller to penetrate the vitreous humor. The preferred microparticle is helical. The microparticle is preferably constructed by the combination of glancing angle deposition technique and the fusion of the slippery liquid layer. The magnetically propulsion in the vitreous humour relies on the matched size of the propeller to the collagen network of the vitreous, and the anti-adhesion coating of the collagen fibre bundles. The clinical optical coherence tomography observed the displacement of the slippery micropropellers through the vitreous to the macular area on the retina. The slippery micropropellers realized the controllable massive movements to the retina in 30 mins, while exerting the travelling distance of above one centimetre. Therefore, the injection of the slippery micropropellers, the magnetically-powered controllable propulsion in the vitreous, and the optical coherence tomography imaging technique, constitute an intact method for rapid targeted ocular delivery, providing a promising approach towards ophthalmologic applications.

We report the first microparticles that actively propel through the vitreous humor and reach the retina. The particles are helical in shape with the diameter matches the mesh size of the biopolymeric network of the vitreous, and a slippery surface coating on the particles minimizes the interaction to the collagen bundles. The latter is inspired from the Nepenthes pitcher plant, which render the insects fall pray by creating a slippery liquid layer on their peristome.^{[[28-30]]} The natural mechanism of the slippery liquid layer utilizes the inherent dynamic and the self-healing nature of liquids to prevent the adhesion from the biomass, promoting the development of the man-made slippery liquid layers mainly based on the non-toxic silicone oil and fluorocarbons.[[³¹⁻³²]] The particles have a magnetic part that possesses a finite magnetic moment. Under the wireless actuation of an external magnetic field, the slippery micropropellers not only show controllable propulsion, but a massive amount also exhibit long-distance locomotion through the complete eye ball and reach the retina, observed by the optical coherence tomography (OCT) imaging. The travelling distance in eye is beyond centimetre scale within 30 min. We expect that the whole operating procedure (FIG. 1), including the intravitreal injection, the fast long-range self-propulsion, and the non-invasive monitoring via a clinically approved instrument, brings the targeted delivery approach one step further towards ophthalmological therapies.

### BRIEF DESCRIPTION OF THE FIGURES

The invention is illustrated in greater detail with the aid of schematic drawings:

FIG. 1 Schematic of the targeted delivery procedures of the slippery micropropellers through the vitreous humor in three steps: 1) Injection of the micropropellers into the vitreous. 2) The magnetically-driven long-range propulsion of the micropropellers in the vitreous towards the retina. 3) The observation of the micropropellers at the target region on the retina by OCT.

FIG. 2 Fabrication and characterization of the slippery micropropellers. (a) Schematic of the fabrication process. (b and c) Scanning electron microscope (SEM) images of the slippery micropropellers. (d) Fourier transform infrared spectroscopy (FTIR) of the micropropellers without coating (above) and with slippery coating (below). The insert images show the contact angles of the wafers with bare and coated microhelices array, respectively.

FIG. 3 Controllable movement of the slippery micropropellers in the vitreous humor. (a) Time-lapse microscopic images showing the incomplete rotation of a bare micropropeller for one cycle in the vitreous. (b) Time-lapse images showing the complete rotation for one full round of a bare micropropellers in the vitreous. The dot in each frame indicates the peak position of the helix. (c) Passive diffusion coefficients of bare silica particles and slippery-layer functionalized particles in the vitreous. (d) Controllable propulsion of slippery micropropellers in the vitreous under the magnetic field. The lines indicate the trajectories of the propellers. (e) Dependence of the propulsion velocity of the slippery micropropeller on the driving frequency of the magnetic field in the vitreous and water.

FIG. 4 Characterization of the movement of the slippery micropropellers in the vitreous. (a) Two typical trajectories of the micropropellers and their corresponding dynamic velocities (inset). (b) Histograms the dynamic velocities of the microhelices in the vitreous and 25% glycerol. (c) The trajectories of the slippery micropropellers in the vitreous show the oscillation of the propulsion direction. The inset shows the distribution of the Swing Angle over 100 μm distance of micropropellers in the vitreous (dark) and in 25% glycerol (white), respectively (n>60).

FIG. 5 Movement of the slippery micropropellers in the complete eye ball. (a) Schematic illustrating the massive movement of the slippery micropropellers in the vitreous. Passive fluorescent particles are injected together with the micropropellers to mark the injection position. (b) Fluorescent image showing the passive particles remain at the injection spot in the vitreous. (c) Autofluorescent image of the retina near the macular. (d) The particle counts by the 3D reconstruction of OCT scans, showing the distribution of the micropropellers in corresponding dashed line labeled area in (c). (e and f) OCT images of x and y scans, respectively, near the propellers landing zone. The dashed lines circle the micropropellers near the retina. The scanning planes are indicated as long arrows in (c). (g) OCT images of the y scan away from the propellers landing zone.

Supplementary FIG. 1 Scanning electron image of the bare micropropellers.

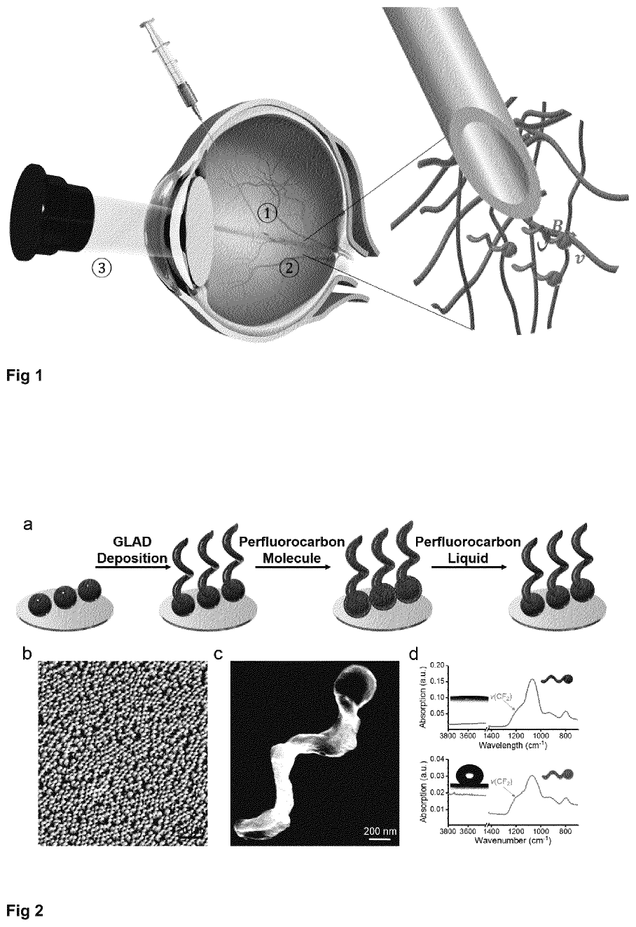

Supplementary FIG. 2 Schematic of the experimental method to confirm the movement of the propellers in the vitreous. The observed site by the microscope is 3 mm away from the initial injection spot.

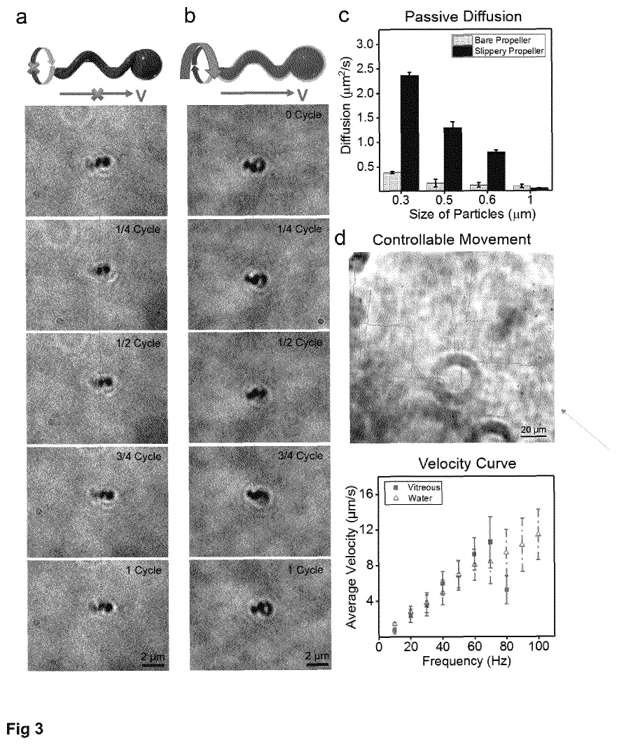

Supplementary FIG. 3 (a) Microscope image shows that only a few perfluorocarbon molecule-functionalized micropropellers are able to move in the vitreous. (b) Microscope image shows that a large population of the slippery micropropellers cross the boundary of the buffer and vitreous and continuously move in the vitreous. Coloured lines indicate the trajectories of the micropropellers. (c) The trajectory of the micropropeller propelling from buffer solution to the vitreous.

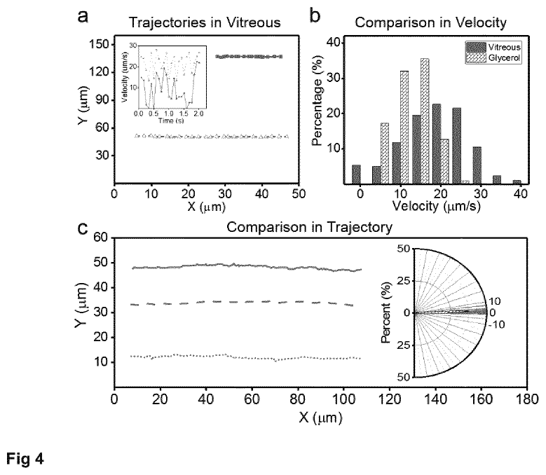

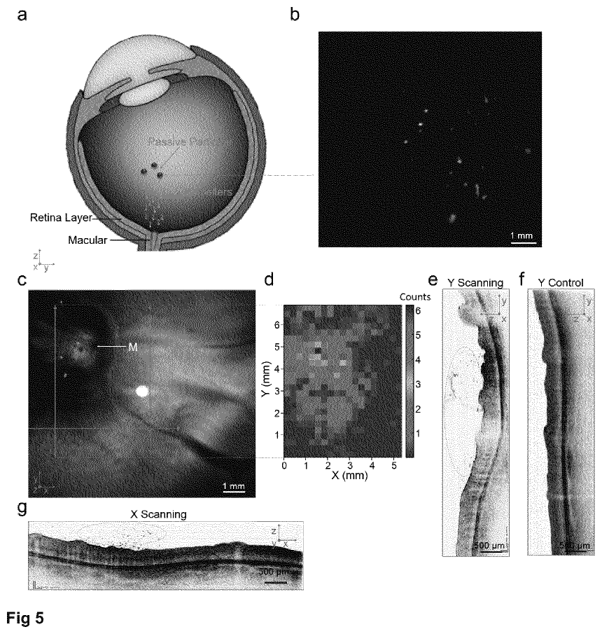

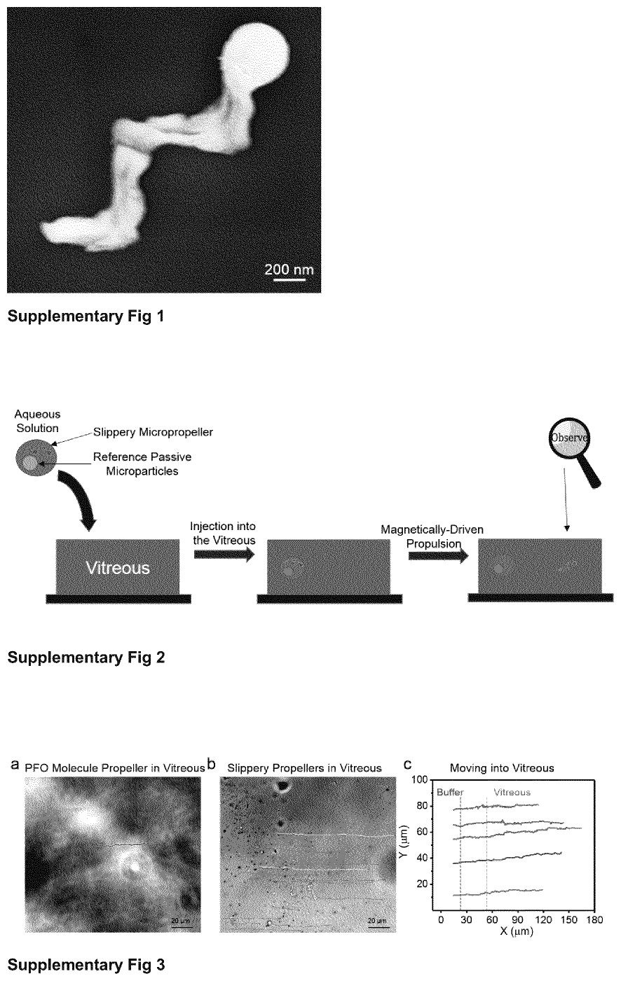

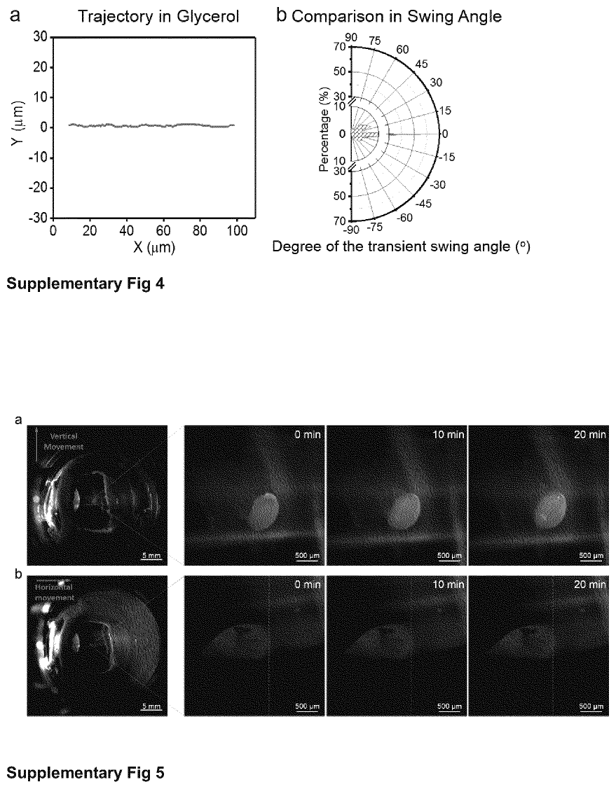

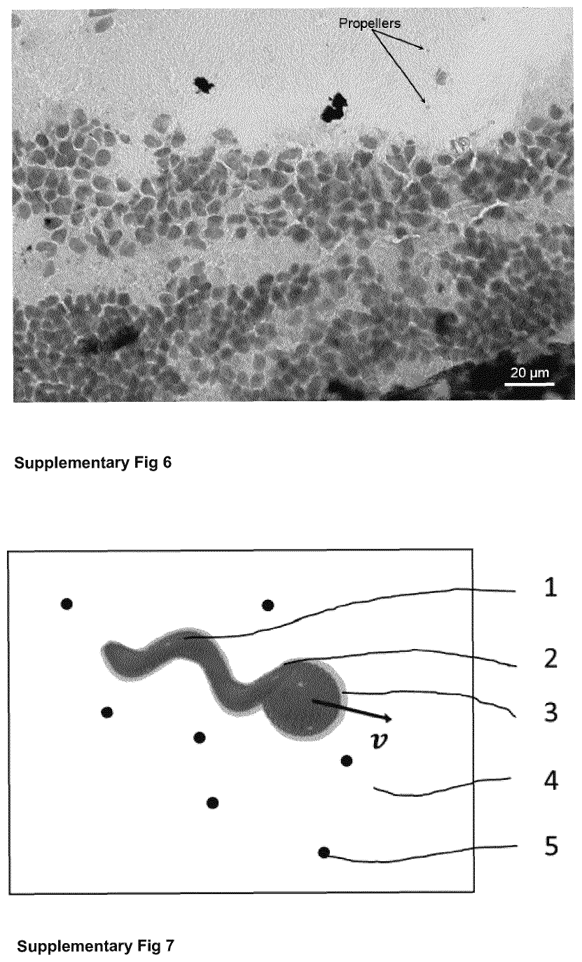

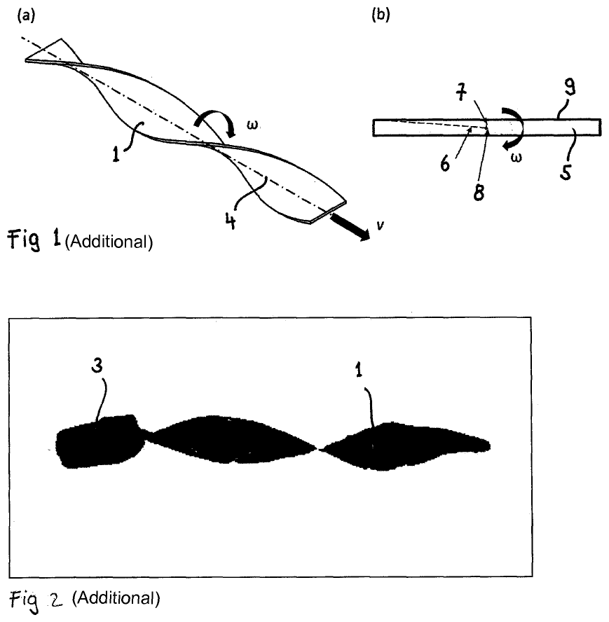

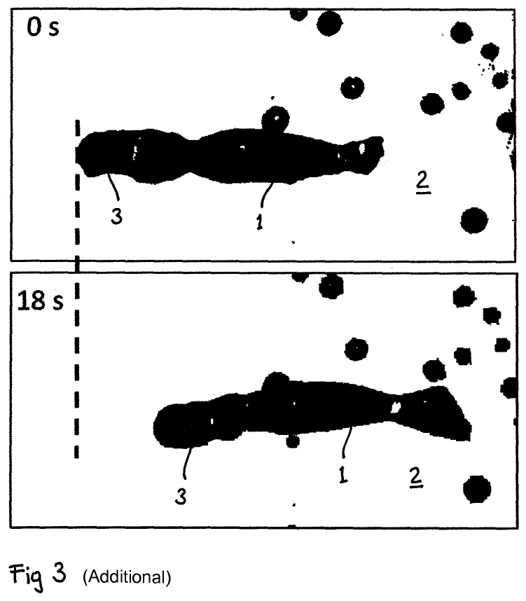

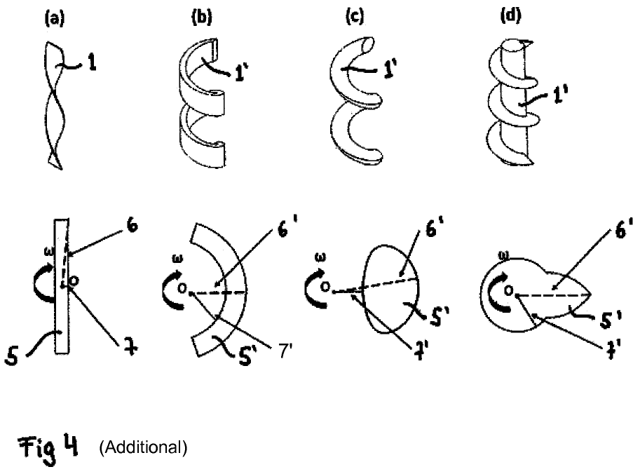

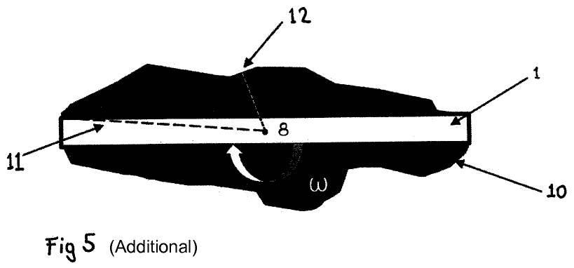

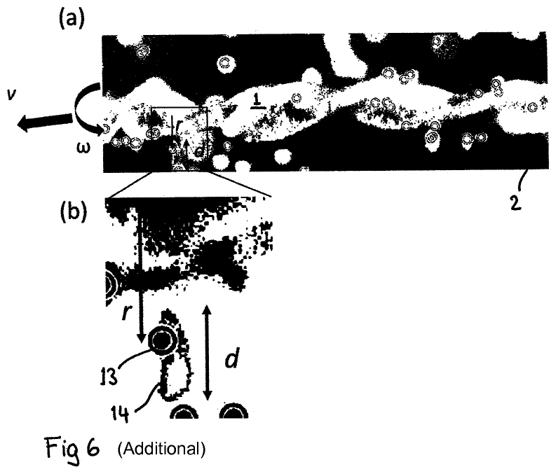

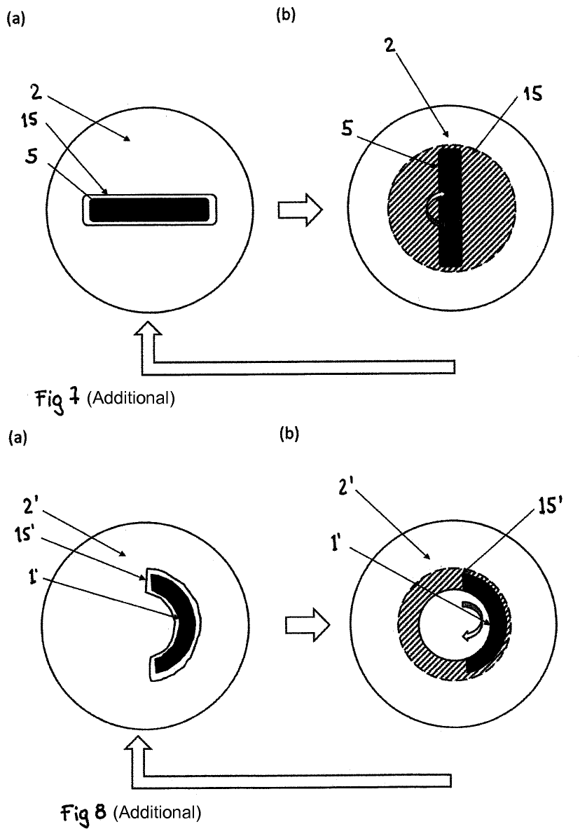

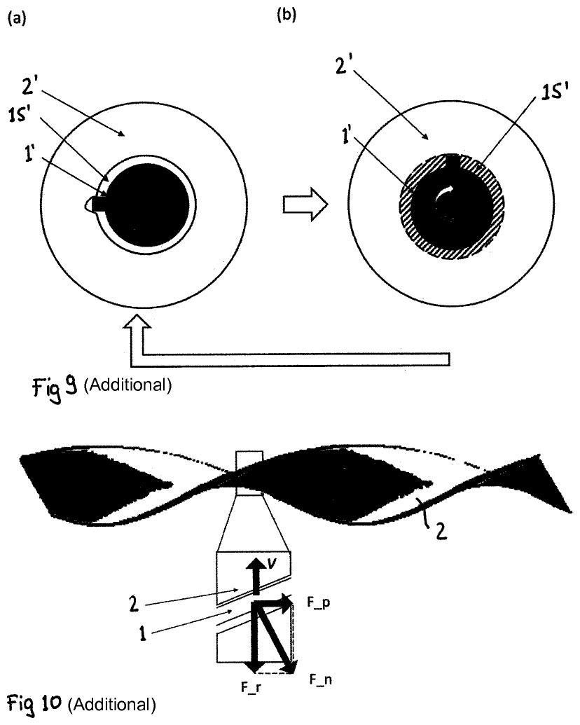

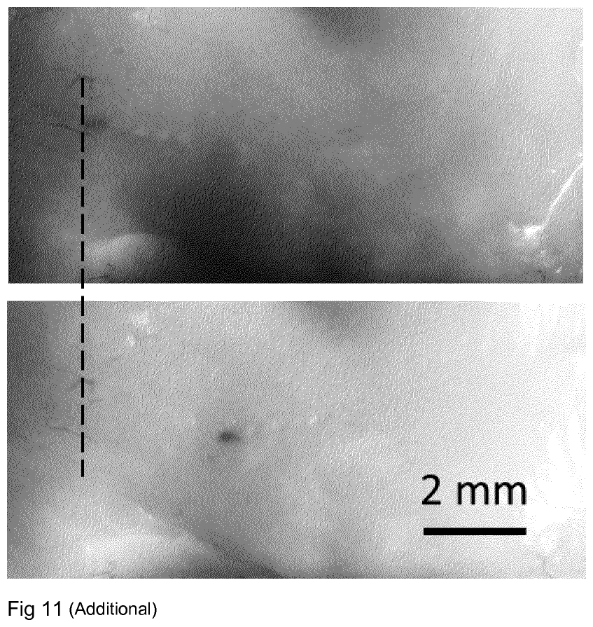

Supplementary FIG. 4 (a) Trajectory of the slippery micropropellers in glycerol solution. (b) The comparison of the transient swing angle for the movement of the slippery micropropellers in the vitreous (hashed) and in 25% glycerol.

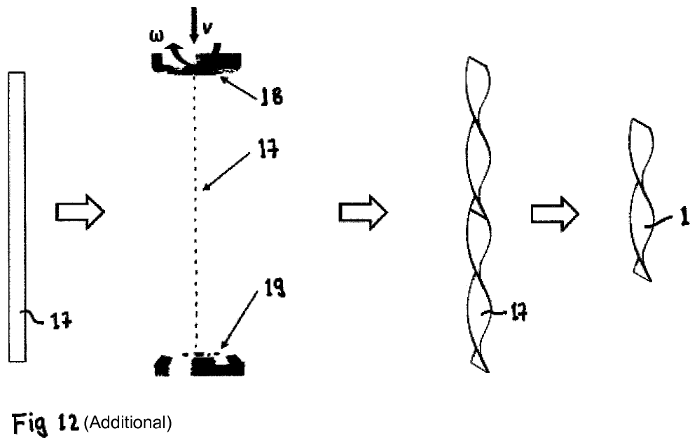

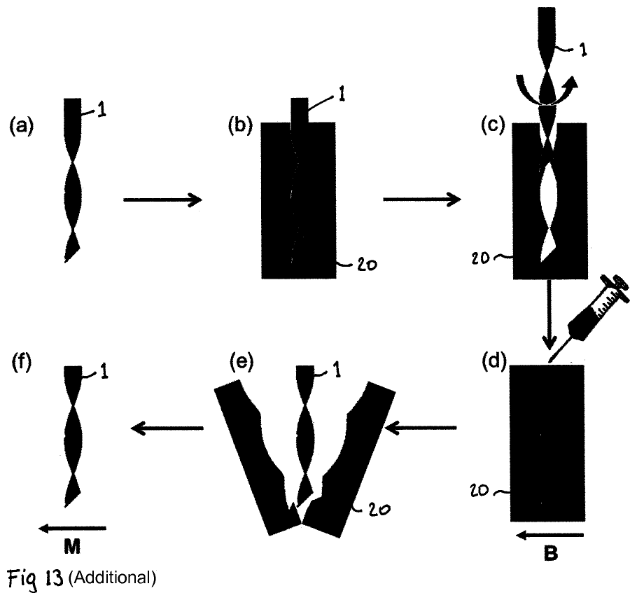

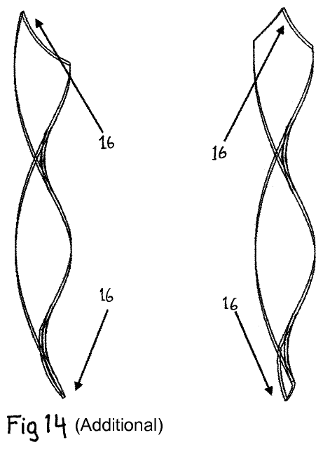

Supplementary FIG. 5 Time-lapse fluorescence fluorescent images showing the vertical (a) and horizontal (b) mass movement of the quantum dots functionalized slippery micropropellers in the vitreous of the eye.

Supplementary FIG. 6 The histology image showing the location of the propellers in the vicinity of the retina after the injection of the slipper propellers into the centre of the eye and moved towards the retina under the external rotating magnetic field at a frequency of 70 Hz and a strength of 8 mT for 1 h.

Supplementary FIG. 7 Schematic of a slippery micropropeller according to the invention moving through a biomaterial. The particle (1), coated with at least one solid layer (2) and at least one liquid layer (3), diffuses or active transports through a medium (4). A stabilizer (5) can be added in the solution to keep the particle (1) dispersed.

### RESULTS

Fabrication and Surface Coating of the Slippery Micro Propellers.

The fabrication of the slippery micropropellers consists of two main steps: the preparation of helical microstructures and the coating of a slippery layer onto the microhelices (FIG. 2a). The helical microstructures were fabricated by the glancing angle deposition (GLAD) technique, as described previously.[[³³]] See Mark, A. G., Gibbs, J. G., Lee, T.-C. & Fischer, P. Hybrid nanocolloids with programmed three-dimensional shape and material composition. Nat Mater 12, 802-807, doi:10.1038/nmat3685 http://www.nature.com/nmat/journal/v12/n9/abs/nmat3685.html#supplementary-information (2013). The helices consist of Silica as the structural segment and Nickel as the magnetic segment (see the Methods section for details). The resulting microhelices were functionalized with a molecular perfluorocarbon layer by gas phase deposition, and subsequently fused with a slippery perfluorocarbon liquid layer.[[³⁴]] See Zhou, H. et al. Fluoroalkyl Silane Modified Silicone Rubber/Nanoparticle Composite: A Super Durable, Robust Superhydrophobic Fabric Coating. Advanced Materials 24, 2409-2412, doi:10.1002/adma.201200184 (2012). Finally, the slippery microhelices were released from the wafer and well dispersed into aqueous media. The fusion of the perfluorocarbon liquid onto the perfluorocarbon molecule-functionalized microhelices retained the full coverage and durable of lubricating liquid surface.

The scanning electron microscope image in FIG. 2b confirms the high-fidelity mass production of the microhelices based on the GLAD method. The enlarged SEM image FIG. 2c shows a typical helix geometry with a length of 2 μm and a silica head of 500 nm in diameter, which matches the mesh size of network in vitreous. By the comparison with the bare helix in Supplementary FIG. 1, the immobilization of the slippery liquid layer maintain the geometry of the bare microhelix, ensuring the efficient propulsion upon the external rotating magnetic field. To evaluate the surface energy of the microhelices, the measurement of water contact angle was conducted on the wafer with microhelices array. The inserted images in FIG. 2d show that the contact angles of the bare microhelices array and the perfluorocarbon liquid layer-functionalized microhelices are 7° and 145°, respectively. The large increase of the water contact angle verifies the effective enhancement of the hydrophobicity and decrease of the surface energy of the micropropellers. Furthermore, comparing spectrum of the bare microhelices by the Fourier-transform infrared spectroscopy (FTIR), the spectrum of the slippery microhelices in FIG. 2d reveals the characteristic peaks of CF2 group at 1199 cm⁻¹,[[³⁵]] see Mihaly, J. et al. FTIR and FT-Raman spectroscopic study on polymer based high pressure digestion vessels. Croatica chemica acta 79, 497-501 (2006), confirming the functionalization of perfluorocarbon materials.

Controlled Propulsion in the Vitreous

Porcine eyes were used as the model for human eyes due to their similar anatomy and properties of the vitreous.[[³⁶]] See Lee, B., Litt, M. & Buchsbaum, G. Rheology of the vitreous body: Part 2. Viscoelasticity of bovine and porcine vitreous. Biorheology 31, 327-338 (1994). The micropropellers were driven wirelessly via a rotating magnetic field with a homogeneous magnitude of 8 mT. The comparable size (the silica head of 500 nm) of the micropropellers to the mesh size of vitreous, allows for their movement through the network of the vitreous. And the slippery liquid layer facilities the micropropellers to repel the adhesion of the vitreous, both effects result in the high intravitreal propulsion of the slippery propeller through the network of the vitreous. In order to confirm that the propulsion occurred in vitreous, the observation strategy was designed as illustrated in Supplementary FIG. 2. The slippery micropropellers and the silica microparticles as reference were suspended in aqueous solution. The mixture suspension was injected into the vitreous, and the observation was conducted in the area at least 3 mm away from the reference particles to confirm that the propulsion in vitreous other than the injected aqueous solution. Meanwhile, the behavior in vitreous of the bare micropropellers and the perfluorocarbon-molecule-functionalized propellers upon the rotating magnetic field was also investigated as control. The time-lapse images in FIG. 3a show the rotation of the bare micropropellers in one cycle, the bare propellers are unable to perform a complete rotation and exhibited the wobbling motion, rotating around the axis with a misalignment angle (FIG. 3a, Supplementary Movie 1), indicating the perfluorocarbon functionalization is essential for the propulsion in vitreous by reducing the adhesion between the microhelices and the polymeric network in the medium.

In contrast, the micropropellers functionalized with perfluorocarbons endow a complete rotation and display obvious displacement of an average 200 nm in one cycle, which basically fits the pitch of the microhelix (FIG. 3b). Additionally, only a small ratio of the micropropellers that functionalized with only perfluorocarbon molecules accomplished the propulsion in the vitreous (Supplementary FIG. 3a). The functionalization of the slippery fluorocarbon-liquid layer displayed advanced propulsion properties in the vitreous compared with those with only the fluorocarbon-molecule coating. When the particles are coated with both solid and liquid layers on the surface, they not only achieved efficient propulsion in a high percentage, but also showed long-range propulsion in the eye for centimeter scale displacement. It clearly suggests the advantages of the slippery fluorocarbon-liquid functionalization including the defects-free coverage, considerable pressure-stability, and the self-healing effect.[[²⁹]] See Wong, T.-S. et al. Bioinspired self-repairing slippery surfaces with pressure-stable omniphobicity. Nature 477, 443-447, doi:http://www.nature.com/nature/journal/v477/n7365/abs/nature10447.html#supplementary-information (2011). The time-lapse image of Supplementary FIG. 3b, captured from Supplementary Movie 2, illustrate that the large population of the slippery micropropellers move from aqueous buffer across the boundary into the vitreous upon the magnetic fields with a strength of 8 mT and a frequency of 70 Hz. The observed traveling distances under the optical microscope exceed 5 mm. The corresponding track lines in Supplementary FIG. 3c shows the aggravate vibration during the movement of the slippery propellers from the aqueous buffer to the vitreous. Moreover, the perfluorocarbon liquid layer is considerabl[y] durable and the slippery micropropellers are able to move in the vitreous after the storage for more than one month.

The movement directionality of the slippery micropropellers can be controlled through manipulation of the magnetic field from the Helmholtz coil. The time-lapse image in FIG. 3d displays the reversible magnetic navigation of the slippery micropropellers to follow the predetermined path in vitreous. The dependence of the velocity of the slippery micropropellers in vitreous and water on the external magnetic frequency was also investigated. As shown in FIG. 3e, the average velocity of the slippery micropropellers in water increases from 1.4 μm/s at 10 Hz to 11.4 μm/s at 100 Hz. The propulsion in vitreous exhibit a similar trend over the 10 to 70 Hz range from 0.7 μm/s to 10.6 μm/s, while the step out frequency was in 70 Hz. Beyond the step-out frequency the velocity of the slippery propeller decreased dramatically. These results imply that the high viscosity of vitreous has an impact effect on the step-out frequency of the slippery micropropellers. Such dependence on the magnetic frequency provides potential on-demand velocity of the slippery propeller upon the modulation of external magnetic frequency.

Propulsion Behavior In Vitreous

The heterogeneity of the vitreous results in the unique locomotion behavior of the micropropellers compared with that in other model viscoelastic media. For example, two typical trajectories of the slippery micropropellers in vitreous, straight and wobbling motions were found (FIG. 4a). Particularly, we observed the phenomenon that the propellers are stuck and restart the movement during the wobbling motions in the vitreous, which is a unusual movement behavior for the helix-shape propellers in other homogeneous viscoelastic media.[[²⁵]] See Schamel, D. et al. Nanopropellers and Their Actuation in Complex Viscoelastic Media. ACS Nano 8, 8794-8801, doi:10.1021/nn502360t (2014). By analyzing the dynamic velocity of the two trajectories, the slippery micropropellers exist the transient velocity of 0 μm/s during the propulsion in the vitreous.

To investigate locomotion behavior of the slippery micropropellers in vitreous, glycerol solution with the same dynamic viscosity as the vitreous was employed as a model viscous media. The trajectory of the helix propeller in glycerol solution shows less wobbling than that in the vitreous at the same rotating magnetic field (Supplementary FIG. 4a). To further analysis these observations, the statics of the dynamic swinging angles during their propulsion were conducted to quantify the propulsion behavior in vitreous, and the results in Supplementary FIG. 4b illustrates the 75% of the swinging angle in glycerol solution are in range of −10° to 10°, and the swing angle below −30° and above 30° is just 16%, while the swing angle below −30° and above 30° for the propulsion in vitreous is 40%. Such nearly 3-fold increase of the angle demonstrates the heterogeneity of the vitreous. For the intensive dynamic swing angle in vitreous, it has been reported that the significant increase of the rotational diffusion of the light-powered Janus propellers in viscoelastic fluids due to the Weissenberg number,[[³⁷]] see Gomez-Solano, J. R., Blokhuis, A. & Bechinger, C. Dynamics of self-propelled Janus particles in viscoelastic fluids. Physical review letters 116, 138301 (2016).it is possible to explain the propulsion in vitreous based on the theory.

The dynamic velocities in vitreous and glycerol were also compared to investigate the intravitreal movement behaviour of the slippery micropropellers. As shown in the histograms representation in FIG. 4b, the dynamic velocity in glycerol solution display a narrow distribution, ranging from 5-20 μm/s. In contrast, a wide distribution of the dynamic velocity in vitreous from 0-40 μm/s was observed. More interestingly, more than 5% of the dynamic velocity is 0 μm/s, suggesting that it gets stuck in the media, which is an unusual movement behavior for the micropropellers that commonly not observed in any Newtonian fluids or even many viscoelastic model fluids.

Although the intensive swinging angle in vitreous, the slippery micropropellers demonstrate negligible deviation during their whole path, which may attribute to the random distribution of the swing angle and the fixed rotational direction of the external magnetic field. The trajectories in FIG. 4c shows the horizontal propulsion (X direction) of the three slippery micropropellers in vitreous with the total travel distance of 100 μm, while their vertical displacement (Y direction) are below 5 μm. The dashed line in FIG. 4c show[s] the ultimate direction of the propeller during the travelling movement, and the quantification was also conducted. The white area in FIG. 4c display the quantification of the deviation angle, near 90% are included in the range of −5° to 5°. These data confirm that the magnetic propulsion of the slippery micropropellers is highly directional in the vitreous in spite of the intensive rotation diffusion.

Observation of the Slippery Micropropellers by the OCT System

One major obstacle to apply artificial micro/nanopropellers to practical clinical routines is the lack of suitable imaging technique in vivo.[[³⁸]] See Medina-Sanchez, M. & Schmidt, O. G. Medical microbots need better imaging and control. Nature 545, 25 (2017). To investigate the mass propulsion through a complete eye ball, we initially attempt to observe the controllable mass movement of the slippery micropropellers in vitreous with the aid of the fluorescence-imaging. The slippery microhelix propellers was functionalized with quantum dots (QDs), and then a high concentrated slipper micropropellers suspension was injected into the center of a porcine eye. The time-lapse images in Supplementary FIG. 5 show the massive movement of the fluorescent slippery micropropellers in vitreous under the Helmholtz coil at intensity of 8 mT and frequency of 70 Hz for 20 mins. The cloud-like area indicates the concentrated micropropellers suspension, and the fluorescence clouds in Supplementary FIGS. 5a and 5b changed their shape vertically and horizontally to the axis of the eye as the manipulation of the external rotating magnetic field, indicating the effective population of the slippery microhelices with controllable propulsion in vitreous. It should be noted that the concentrated slippery micropropellers suspension was injected to obtain enough imaging fluorescent signal, which may also lead to intensive aggregation of the propellers and thus decrease the velocity of the propellers.

To investigate the long-range propulsion of micropropellers in a complete eye ball, we utilized a standard technique in the clinical ophthalmology, optical coherence tomography (OCT). As shown in FIG. 5a, a mixture containing the slipper micropropellers and passive fluorescent microparticles were injected into the center of the porcine eye, and subsequently the porcine eye was undergo the rotating magnetic field toward the retina for 1 h[.] Here the fluorescent silica particles were employed as labels for the injection spot to confirm the propulsion of the slippery micropropellers. The OCT-captured fluorescent image in FIG. 5b indicates the passive fluorescent particles are still located in the vitreous area of the eye. In contrast, the 3D reconstruction results exhibits that the slippery micropropellers in eye under the rotating magnetic field results in an intensive location in the retina of the macular area (FIG. 5c-5g). FIG. 5c show the xy horizontal of the retina, and the macular locates at the top left of the image. The corresponding yz orthogonal section in FIG. 5f illustrates that the retina which is far away from macular, being served as a control, displayed negligible spots near the retina. However, both the xz and yz orthogonal section of the retina in macular area display a large number of dark spots close to the retina, implying the controlled movement of the slippery micropropellers reached the retina in macular area under the manipulation of external magnetic field. We further calculated the number of spots in the scanned images to quantify the distribution of the propellers. The corresponding distribution of the spots in the scanning area was exhibited in FIG. 5d exhibits that the major sports were located in the macular area with a diameter of below 6 mm. Besides the OCT results, the histology image in Supplementary FIG. 6 also verify the location of the slippery propellers at the retina under the propulsion for 1 h under the external magnetic field.

It is more than 1 cm from the center of eye to the retina of the eye, indicating the centimeter total travel distance of the slippery micropropellers in eye. Slower velocity (equaling to 5 μm/s) compared with that in the piece of vitreous segment may reflects the increased viscosity in eye.[[¹⁷]] See Servant, A., Qiu, F., Mazza, M., Kostarelos, K. & Nelson, B. J. Controlled In Vivo Swimming of a Swarm of Bacteria-Like Microrobotic Flagella. Advanced Materials 27, 2981-2988, doi:10.1002/adma.201404444 (2015). The similar propulsion behavior in the intact eye is similar as that in the tiny piece of the vitreous. These above data clearly suggests that the slippery micropropellers can be manipulated to the predetermined position in the eye and potentially in other porous biological tissues. OCT provides an optimized strategy to track the propellers non-invasively, suggesting the potential capacity to apply and monitor the microparticles for clinical ophthalmology applications.

Discussion

In summary, for the first time, we report the active long-range propulsion of microparticles through porous biological tissues. The propulsion is enabled by magnetic helical micropropellers that has a diameter similar with the mesh size of the media and a slippery coating to minimize the adsorption with the media. Specifically, helical micropropellers of 2 μm in length with roughly 1 μm² surface coating are able to propel in porcine vitreous at a maximal speed of beyond 10 μm·s⁻¹. Clinical standard OCT system can monitor the movements of the particles and confirm their arrival on the retina in 30 min. The rapid and long-distance intravitreal propulsion in the eye, together with the monitoring by a clinically approved non-invasive method, shed light in bringing active microparticles towards clinical applications.

Future promises for the propeller to the clinical ocular therapeutics can be accomplished by combining advanced propeller designs with diverse medical protocols. For example, it could be possible to load various therapeutic agents, such as drugs, inductive heating materials, radioactive materials; and also imaging agents, including image contrast enhancers and fluorophores to the slippery micropropellers for the rapid delivery towards the targeted and hard-to-reach regions in the human or animal body. The driven force from the large population of the propellers may form a macroscale mechanic force to the tissues, which may be large enough for a minimally invasive surgery. These current capabilities and potential promises of the slippery micropropellers would be expected with the injectable delivery and real-time monitoring and feedback platform, which may creates new possibilities for future medicine.

Methods

Fabrication Process of the Slippery Micro Propellers

The bare microhelixes were prepared though the GLAD deposition as our previous report. A Langmuir-Blodgett monolayer of silica particles with average size of 500 nm was first sprayed on the silicon wafer to serve as a seed layer. Nickel was initially deposited onto the surface of the silica particle seeds, and silica was subsequently evaporated onto the nickel segments of the microhelix.

In this case, the particle has a helical shape. The rotation of the chiral structure results in propulsion at low Reynolds number in the medium. However, the particle can also be not chiral when only its shape is considered, but when taking the magnetic moment of the particle into account, then it is chiral. This situation is defined as “modified chiral” in the current application.

In order to decorate the slippery liquid layer onto the microhelixes, the wafer containing the microhelix patterns was treated with the oxygen plasma at 200 mW for 15 s, and then the activated wafer was incubated with 20 μL of perfluorocarbon silane under vacuum for 20 mins, followed by the heating at 85° C. at atmospheric pressure for 1 h. The microhelix was then immersed into the perfluorocarbon liquid under the mechanical shaking overnight. After the resining with acetone for one time, the wafer was blow with nitrogen gas until no liquid on the wafer.

To prepare the QDs-functionalized slippery micropropellers s, the oxygen plasma-treated microhelix was immersed into 1.5% (v/v) APTES (95%, Sigma-Aldrich) in toluene solution. The wafer was then incubated with 0.25 mg/mL CdSe 560 (Sigma-Aldrich) in toluene overnight. After that the wafer was sequentially resin with toluene, acetone, and water. To protect the QDs of the microhelix from the oxidation in the following oxygen plasma treatment, an Al₂O₃ adhesion layer with thickness of 10 nm was deposited onto the microhelix wafer through atomic layer deposition (ALD) for 100. The ALD and QDs-functionalized wafer with microhelix patterns went through the same procedure as the bare microhelix wafer.

The magnetic property of the microhelix was tested through SQUID method to the microhelix wafer at 300 K by the usage of a Quantum Design MPMS magnetometer. Prior to the experiment in motion in vitreous, the wafer with the slippery micropropellers was magnetized by an electromagnet with strength of 1.7 T.

Characterization Techniques

The contact angle was performed on a in a Dataphysics OCAH 230. The samples were prepared by dipping 3 μL of the water droplet on the different samples including the bare wafer, the bare wafer with microhelix patterns, the slippery wafer, and the wafer with slippery microhelix patterns. The results were displayed as the average value of three droplets on the samples. The scanning electronic microscope (SEM) characterization was conducted with a Zeiss Ultra 55 instrument at an operating voltage of 5 keV. A drop of the sample suspension was dropped on a silicon wafer for the test of the slippery micropropellers s. Fourier transform infrared attenuated total reflection (FTIR-ATR) analysis was conducted with a Bruker Vertex 70V in the single reflection mode 45°.

Preparation of the Vitreous and Propulsion Experiments Under the Microscope

The vitreous for the propulsion of the slippery micropropellers at the microscale was directly obtained by cutting from the porcine eyes. In order to prepare the samples, the vitreous with volume of rough 10 μL was firstly lay on one glass coverslip with a geneframe (Thermo Scientific). After that 2 μL of the PBS buffer containing micropropellers was then injected into the vitreous. After that the sample was placed into the center of the magnetic Helmholtz coil attached microscope (Zeiss Observer). The observation was performed at the site where is far away from the passive silica particles to ensure the propulsion in vitreous. The Image J was employed to extract all the frames of the video and analysis the movement behavior of the slippery micropropellers in vitreous including the various trajectories, average velocities under different manipulation parameters, dynamic velocity, the swinging angle, and deviation angles.

Fluorescent Mass Movement In Vitreous at the Macroscale and Experimental Set Up

The characterization of the mass movement in vitreous at the macroscale involve in the assembly of the Helmholtz coil and fluorescent stereoscope set up, the treatment of the porcine eye, and the injection of the concentrated slippery micropropellers. To trigger and observe the macroscale mass movement of the slippery micropropellers in eye, the water cooling Helmholtz coil, a fluorescence filter-installed stereoscope, and UV light was assembled together. The Helmholtz coil was fixed in the plate of the stereoscope, the filter and the camera was vertically above the Helmholtz coil and a UV light from the lamp was horizontally irradiated into the center of the Helmholtz coil.

For the treatment of the porcine eye, the porcine eyes was firstly put in a plastic plate to expose the lens, and a 1% agar suspension with temperature of roughly 45° C. was poured into the plate, and the following cooling procedure to fix the eye in the plate. The top segment of the sclera, the partial plastic plate and the agar gel close to lens was cut by the scalpel to expose the vitreous to the fluorescence stereoscope and the lens to the UV light to the lens. For the injection of the propeller suspension into the vitreous, the microhelix wafer with rough total size of 36 mm² was cut into small pieces and released into the 50 μL aqueous solution, after sonication for 3 mins 25 μL of the suspension was immediately injected into the center to the eye. Then the plate with fixed eye immediately transferred to the Helmholtz coil and the UV light was manipulated to irradiate to the vitreous of eye through its lens. At last, the Helmholtz coil was launched to power the movement of the slippery micropropellers and the camera in the stereoscope recorded the massive movement at the exposure time of 10 s.

Optical Coherence Tomography

The microhelix wafer with rough total size of 15 mm² incubated into the 100 μL aqueous solution with passive 20 μm silica microparticles (Sicastar®-greenF, micromod Partikeltechnologie GmbH), and followed by sonication for 3 mins. The 100 μL of the mixture was immediately injected into the center of the porcine eye by using a pipette. The eye was then lay to the center of the Helmholtz coil toward the direction toward the retina with intensity of 8 mT and frequency of 70 Hz for 1 h. The resulting porcine eye was fixed at a shelf and the OCT instrument was used to image the fluorescence silica particles and the slippery micropropellers at the retina.

In one embodiment of the invention a micropeller in vitreous is exposed to a rotating magnetic field with strength of 8 mT and a frequency of 6 Hz. In another embodiment, a micropeller in vitreous is exposed to a rotating magnetic field with strength of 8 mT and a frequency of 70 Hz. A micropeller according to the present invention may show controlled motion in vitreous under the rotating magnetic field with strength of 8 mT and frequency of 70 Hz. A change of the direction of the micropeller can be conducted by the manipulation of external rotating magnetic field.

Propeller and Method in which a Propeller is Set into Motion

Description

### FIELD OF THE INVENTION

The invention concerns a method in which a propeller is set into locomotion relative to a medium which at least partially surrounds the propeller, wherein an actuator induces a rotation of the propeller relative to the medium and about a rotational axis of the propeller, and wherein the propeller converts its rotational movement into locomotion of the propeller relative to the medium. It moreover concerns a helical or modifiedly helical propeller for converting rotational movement of the propeller into locomotion of the propeller relative to the medium. Furthermore, the invention concerns methods of producing the propeller.

### BACKGROUND OF THE INVENTION

In many applications in medicine and biology it can be of advantage to be able to penetrate biological media, including biological fluids and soft tissues. For example, in minimally invasive procedures, such as the targeted delivery of substances or minimally invasive surgical procedures, it can be desirable to move a small untethered device to penetrate the medium, because such method potentially is less invasive and provides better control than methods that use larger or tethered devices.

Small untethered devices have been reported in the literature. For example, A Ghosh and P Fischer in “Controlled Propulsion of Artificial Magnetic Nanostructured Propellers,” Nano Letters, vol 9, pp 2243 to 2245, 2009 and in the supporting information published with this paper demonstrate that the rotation of a cork-screw-like shape can produce forward propulsion in a fluid. The rotation is effected by a rotating magnetic field. This concept is also described in U.S. Pat. No. 8,768,501 B2. A swimmer with a slightly different shape is disclosed in the publication of L Zhang, XXXX, L X Dong, B E Kratochvil, D Bell, and B J Nelson, “Artificial bacterial flagella: Fabrication and magnetic control, “Applied Physics Letters, vol 94, p 064107, 2009. This swimmer, too, is driven by rotating magnetic field. K lshiyama, M Sendoh, A Yamazaki, and K I Arai in “Swimming micro-machine driven by magnetic torque,” Sensors and Actuators A: Physical, vol 91, pp 141 to 144, 2001 describe a screw, several millimetres in length, that penetrates a bovine tissue (meat) sample when brought into rotation by a rotating magnetic field.

T Qiu, J Gibbs, D Schemel, A Mark, U Choudhury, and P Fischer in “From Nanohelices to Magnetically Actuated Microdrills: A Universal Platform for Some of the Smallest Untethered Microrobotic Systems for Low Reynolds Number and Biological Environments,” Small-Scale Robotics, From Nano-to-Millimeter-Sized Robotic Systems and Applications, vol 8336, I Paprotny and S Bergbreiter, 1st ed Berlin: Springer, pp 53 to 65, 2014 describe the manufacture of a cork-screw-like propeller by means of a glancing angle deposition method (GLAD) and of a propeller that more resembles a conventional screw by means of micro injection moulding. They also describe locomotion of the propellers in agarose gel when the propellers are actuated by means of a rotating magnetic field.

In all of the above disclosures, the propeller has a part with a permanent magnetic moment orthogonal to its long axis or the propeller is attached to a permanent magnet. Application of an external rotating magnetic field exerts a torque that spins the untethered propeller and causes its translation through a medium.

WO 2008/090549 A2 discloses a medical device for insertion into an organ of a patient that can be set into repetitive motion by an external magnetic field. WO 2016/025768 A1 discloses nanoparticles that can move along the gradient of a magnetic field originating from permanent magnets or electromagnets. The nanoparticles have a high tendency to attach to targeted cells, and an electric field can be applied to the nanoparticles to generate actions that are sufficient to cause death of the targeted cells. WO 2011/073725 A1 discloses a handheld automated biopsy device with a drill-like tip. The device can be brought into rotation by an actuator.

EP 2 674 192 A1 discloses a medical implantable device that can be implanted into a human or animal body. It comprises to intertwined helical wires, one of which will upon rotation be screwed into the tissue. US 2012/0010598 A1 discloses a catheterization system that is provided with an external thread and can be advanced into a bodily passageway by means of rotation. US 2009/0248055 A1 discloses a tissue penetrating surgical device. A distal tip of the device is at least partly covered by a fabric and the device can drill into the tissue by means of rotating the fabric.

It can be challenging to further miniaturise existing devices. Moreover, it has proven difficult to obtain propulsion in viscoelastic media with existing devices. The known cork-screw-like shapes work well in viscous liquids such as water and glycerol and in elastic solids such as agarose and meat. However, many important tissues in the biomedical domain are neither purely viscous fluids nor purely elastic solids. Rather, they are viscoelastic media that exhibit the combined properties of both a liquid and a solid. The inventors have found that the known propeller shapes can be inefficient in viscoelastic media.

### Problem to be Solved by the Invention

It is an objective of the present invention to provide an improved method in which a propeller is set into locomotion relative to a medium which at least partially surrounds the propeller, wherein an actuator induces a rotation of the propeller relative to the medium and about a rotational axis of the propeller, and wherein the propeller converts its rotational movement into locomotion of the propeller relative to the medium. It is another objective of the present invention to provide an improved helical or modifiedly helical propeller for converting rotational movement of the propeller into locomotion of the propeller relative to the medium. Also it is an objective of the present invention to provide an improved propeller. It is a further objective of the invention to provide improved methods of producing the propeller. It is achievable with the present invention to address one or more of the afore-mentioned difficulties in the prior art.

### Solution According to the Invention

In one aspect of the invention, the problem is solved by providing a method in which a propeller is set into locomotion relative to a medium which at least partially surrounds the propeller. An actuator induces a rotation of the propeller relative to the medium and about a rotational axis of the propeller, and the propeller converts its rotational movement into locomotion of the propeller relative to the medium. The aspect ratio of at least one cross section of the propeller—which cross section is a cross section related to the propeller's rotational axis—is 3 or more.

The inventors have found that such large aspect ratio can considerably increase propulsion, in particular in viscoelastic media. Without being bound to a particular theory, the inventors believe that the invention exploits a newly discovered propulsion mechanism that employs an elastic deformation of the medium by the propellers rotation. A large aspect ratio can induce a large deformation and thus strong propulsion.

In the context of the present invention, the term “propeller” refers to a propelling structure that can effect locomotion of itself or the load attached to itself relative to a medium. In the context of the present invention, the cross section's “aspect ratio” is the largest radius of the cross section divided by the smallest radius of the cross section, the radii extending from the cross section's centre to a point of the circumference of the cross-section. The cross section's centre is the point where the axis to which the cross section is “related” pierces the cross section. The cross section moreover is perpendicular to the axis to which it the cross section is “related”. The circumference of the cross section is the outer boundary of the cross section. Accordingly, if the cross section is related to the propeller's rotational axis, the radii for determining the aspect ratio extend from the point where the rotational axis perpendicularly pierces the cross section to a point of the circumference of the cross section. Likewise, if the propeller is a helix (see below) and the cross section is related to the propeller's helical axis, the radii for determining the aspect ratio extend from the point where the helical axis perpendicularly pierces the cross section to a point of the circumference of the cross section.

In another aspect of the invention, the problem is again solved by providing a method in which a propeller is set into locomotion relative to a medium which at least partially surrounds the propeller. An actuator induces a rotation of the propeller relative to the medium and about a rotational axis of the propeller, and the propeller converts its rotational movement into locomotion of the propeller relative to the medium. In this aspect of the invention, the aspect ratio of at least one cross section of the rotating body that comprises the propeller and the parts of the medium that due to the rotation the propeller have been severed from the remainder of the medium and rotate with the propeller, which cross section is a cross section related to the rotating body's rotational axis, is 3 or more. Advantageously, with this aspect of the invention it is achievable that the parts of the medium that due to the rotation the propeller have been severed from the remainder of the medium rotate at the same speed as the propeller.

This embodiment of the invention is based on the inventors' discovery that parts of the medium can be severed—for example due to adherence—from the remainder of the medium and as a result rotate with the propeller. The inventors found that such co-rotation can considerably impede propulsion but that by means of a high aspect ratio of the rotating body comprising of the propeller(s) and the co-rotating part of the medium strong propulsion can nevertheless be achieved. Again without being bound to a particular theory, the inventors believe that in the newly discovered propulsion mechanism the propulsion predominantly results from the elastic deformation of the medium that does not co-rotate with the propeller and that as a result, the aspect ratio of the rotating body comprising of the propeller and the co-rotating part of the medium is critical for achieving strong propulsion.

In yet another aspect of the invention, the problem is solved by a helical or modifiedly helical propeller for converting rotational movement of the propeller into locomotion of the propeller relative to a medium which at least partially surrounds the propeller. The aspect ratio of at least one cross section of the propeller, which cross section is a cross section related to the propeller's helical axis, is 3 or more.

In the inventors' experiments, helical and modifiedly helical shapes have proven particularly suitable for achieving propulsion. Moreover, helical and modifiedly helical shapes have proven easy to manufacture.

In the context of the present invention, a propeller is “helical” (further below also referred to as a “helix”) if its three-dimensional shape can be obtained by extending a two-dimensional shape along a curve while rotating the two-dimensional shape. The two-dimensional shape is extended along a curve (further below also referred to as “helical axis”) such that any two cross sections of the propeller, if each cross section is taken perpendicularly to the curve at the point where the curve pierces the cross section, can be brought to coincide with the two-dimensional shape. The helical axis is the curve along which the two-dimensional shape is extended. A helix is chiral. In the context of the present invention, a propeller is “chiral” if its shape is distinguishable from its mirror image's shape; in other words, a propeller has chirality if its image in a plane mirror, ideally realized, cannot be brought to coincide with itself. The propeller can also be chiral by virtue of the orientation of its magnetic moment relative to the body of the propeller; such propellers are defined as “generalized chiral” in the context of the present invention. This includes objects that have an achiral body shape, but possesses a suitably oriented magnetic moment to render the propeller chiral.

In the context of the present invention, “modifiedly helical” (further below also referred to as a “modified helix”) differs from helical in that the two-dimensional shape does not remain the same but changes as it is extended along the curve. The evolution of the two-dimensional shape is continuously differentiable (as opposed to discontinuous or non-differentiable, in a mathematical sense). For example, the two-dimensional shape may be stretched or compressed in one dimension, it may be bent, or it may be shrunken or enlarged proportionally in both dimensions. As a result of the latter for example a section of the propeller or even the entire propeller may have a tapered shape.

In a further aspect of the invention, the problem is solved by a method of producing a propeller, which method comprises the steps of (1) defining a straight helical axis; (2) providing a plate extending along the helical axis, the aspect ratio of at least one cross section (preferably all cross sections) of the plate, which cross section is a cross section related to the helical axis, is 3 or more; and (3) applying to the plate a torque along the helical axis, thereby twisting the plate into helical shape.

This method exploits the inventor's insight that the high ratio of width or length to thickness that is inherent in the definition of a plate can be translated into an aspect ratio of a helix if the helix is twisted by means of applying a torque. The inventors have discovered that this makes for an easy and reliable manufacturing method of a helical propeller with a high aspect ratio.

In yet a further aspect of the invention, the problem is solved by a method of producing a propeller, which method comprises the steps of (1) providing a first structure with a defined geometry; (2) moulding of the first structure in a second material, removing the first structure from the second material to generate a negative replicate of the first structure; (3) injecting a moulding material or moulding materials into the negative mould and curing the moulding material to form a second solid structure under given physical and chemical conditions; and (4) releasing the second solid structure from the negative mould, thereby obtaining the defined propeller.

The invention can advantageously be employed in medical diagnosis and therapy, including endoscopy, biopsy, delivery of drug or implant or radioactive matter, local heat generation. For example, the propeller may carry payloads attached to the propeller and release the drug at the disease location. In tumour therapy, the propeller can propel through the normal tissue to the tumour tissue, if the propeller is made of or comprises a metallic and magnetic material, heat can be generated in the material by inductive heating to kill tumour cells. Also, the propeller can drag a thin flexible tube to the tumour site, and drug can be continuously delivered to the tumour though the tube. Similarly, the propeller can drag an electrode connected with an electric wire and move to a particular region of the brain to measure the neuron electrical signal or apply an electric stimulation.

### Preferred Embodiments of the Invention

Further preferred features of the invention which may be applied alone or in combination are discussed in the dependent claims, description below and the figures.

In a preferred embodiment of the invention, the aspect ratio of at least one cross section, preferably all cross sections, of the propeller, is/are 2 or more, more preferably 3 or more, more preferably 5 or more, more preferably 10 or more, more preferably 20 or more, more preferably 50 or more, more preferably 100 or more; the cross section(s) are related to the propeller's rotational axis or, alternatively, to the propeller's helical axis. This embodiment of the invention exploits the inventors' finding that a particularly high aspect ratio can entail a particularly strong propulsion. The cross section preferably is of a continuous shape.

Preferably, at least one cross section, more preferably every cross-section, of the propeller related to the propeller's rotational axis has a cross-sectional area that is at least 50%, more preferably 100%, more preferably 300%, more preferably 1 000% of the cross-sectional area—in the same cross sectional plane—of the parts of the medium that due to the rotation the propeller(s) have been severed from the remainder of the medium and rotate with the propeller(s). This embodiment exploits the inventors' find that co-rotating medium may impede propulsion and that by limiting the amount of co-rotating material such impediment can be limited.

Preferably, in case of at least one cross section, more preferably in case of all cross-sections, of the propeller the propellers rotational axis passes through the area of the cross section, ie through the inside of the cross section's circumference; the cross section(s) are related to the propeller's rotational axis or, alternatively, to the propeller's helical axis. In other words, in a preferred embodiment of the invention, the rotational or the helical axis passes at least partly through the propeller.

Preferably, at least 20%, more preferably at least 50%, more preferably at least 80%, more preferably at least 95% of the surface area of the propellers has a surface roughness Ra (pursuant to Deutsches Institut für Normung DIN 4760) of less than 3.2 μm, more preferably less than 1.6 μm, more preferably less than 0.4 μm, more preferably less than 0.025 μm, more preferably less than 0.006 μm. With this embodiment of the invention it can advantageously be achieved that the adherence of medium, such as biological tissue, to the propeller, which adherence may impeding locomotion, is reduced. The surface roughness of the propeller is low to minimize the adhesion of the medium on the surface of the propeller.

Preferably, in order to minimize adhesion, the material from the propeller, at least at the surface of the propeller, is a metal, an anti-adhesion polymer and/or a biocompatible polymer. Coating can be applied to the surface of the propeller to minimize the adhesion of the medium onto the surface of the propeller. Special actuation methods that induce large shear on the surface, for instance, sudden start or stop of a large-angle rotation, large-angle oscillation, can be applied to minimize the attachment of the medium. For example, an oscillation of the propeller can be applied with a gradually increased amplitude from 10° to 300° and/or a gradually increased frequency from 0.1 Hz to 10 Hz, before the full rotation of the propeller. Due to the viscoelasticity of the medium, eg shear-thinning effect, the actuation method lowers the required starting torque for full rotation of the propeller.

In a particularly preferred embodiment of the invention, the low surface roughness is achieved by means of an at least partially coating of the surface of the propeller, More preferably the entire surface of the propeller is coated. Preferred coating materials include Teflon, PEG (Polyethylene glycol), Titanium or a combination thereof.

In a preferred embodiment of the invention, the aspect ratio of at least one cross section, preferably all cross sections, of the rotating body that comprise(s) the propeller and the parts of the medium that due to the rotation the propeller have been severed from the remainder of the medium and rotate with the propeller, which cross section(s) is/are a cross section related to the rotating body's rotational axis, is/are 2 or more, more preferably 3 or more, more preferably 5 or more, more preferably 10 or more, more preferably 20 or more, more preferably 50 or more, more preferably 100 or more. This embodiment of the invention exploits the inventors' finding that while co-rotation can impede propulsion, by means of a high aspect ratio of the rotating body comprising of the propeller(s) and the co-rotating part of the medium strong propulsion can nevertheless be achieved. The cross section of the rotating body preferably is of a continuous shape.

The preferred propeller is chiral. More preferably the propeller is helical or modifiedly helical. This embodiment of the invention is based on the inventors finding that chiral and in particular helical and modifiedly helical shapes can be particularly effective for achieving propulsion. Moreover, helical and modifiedly helical shapes have proven easy to manufacture. Preferably, the helical axis is a straight. If the propeller or the propeller is a helix or a modified helix, the rotational axis preferably coincides with the helical axis. The preferred helical or modifiedly helical propeller has a constant pitch.

A preferred propeller has a forward taper on at least one end, more preferably on two opposite ends. In the context of the present invention, a “forward taper” means that the propeller towards an end of the propeller is becoming gradually smaller or thinner. Preferably, the front end of the propeller is provided with a forward taper. In the context of the present application, the “front end” is the leading side of the propeller with regard to the direction of locomotion It is an achievable advantage of this embodiment of the invention that the taper can decrease the area of contact with the medium at the front end of the propeller. It can be achieved that—in particular if the medium has viscoelastic properties—the pressure which the propeller applies on the medium is larger than the tensile strength of the medium.

In one embodiment, the tip of the taper is located on the rotational axis of the propeller, and/or on the helical axis, provided that the propeller is a helix. In another embodiment, the tip is located eccentrically, ie away from the rotational axis, and/or away from the helical axis, provided that the propeller is a helix. Particularly preferably the tip is located near the outer perimeter, with respect to the rotational axis or helical axis of the propeller.

This embodiment can exploit the fact that many media have shear-thinning properties so that a large shear rate can help the forward propulsion of the propeller. As the velocity is the greatest at the outer perimeter of the propeller, a tip located there can achieve the greatest shear rate.

The largest radius of any cross section of the propeller that is perpendicular to the propeller's rotational axis or helical axis preferably is 5 mm or less, more preferably 3 mm or less, more preferably 1 mm or less, more preferably 500 pm or less, more preferably 300 pm or less, more preferably 100 pm, or less, more preferably 50 pm or less, more preferably 30 μm or less.

The smallest radius of any cross section of the propeller that is perpendicular to the propeller's rotational axis or helical axis preferably is 300 μm or less, more preferably 100 μm or less, more preferably 50 μm or less, more preferably 30 μm or less, more preferably 10 μm or less, more preferably 5 μm or less, more preferably 3 μm or less.

The length of the propeller divided by the largest radius of any cross section of the propeller that is perpendicular to the propeller's rotational axis or helical axis preferably is 0.5 or more, more preferably 1 or more, more preferably 3 or more, more preferably 5 or more.

Preferably, the propeller is untethered. In the context of the present invention, “untethered” means that the propeller has no material connection—for example in the form of a wire, a tube or a rod—to the space outside the medium by which the propeller is at least partly, preferably completely, surrounded. Alternatively the propeller is minimally-tethered, whereas the driving torque for the propeller is applied wirelessly, but the propeller is connected to a passive element, for example to pull the end of a tube and/or a wire, which other end is outside the medium, into a particular position inside the medium. The tether can be used for material transportation, signal measurement or stimulation, but the tether is passive that it does not provide[[s]] active driving force or torque to the propeller. Alternatively the propeller is tethered, for example the driving torque of the propeller is input by a string, a wire or a rod, whose rotation leads to the locomotion of the propeller together with the tether.

The rotation of the propeller preferably is induced remotely. In the context of the present invention “effected remotely” means that means that induce the rotation of the propeller are located at a distance from the propeller that is at least 5 times the largest diameter of the propeller in any dimension. In a preferred embodiment of the invention, the rotation of the propeller is induced remotely by means of a magnetic field. Thus, the source of the magnetic field is located at a distance from the propeller that is at least 5 times the largest diameter of the propeller in any dimension, and the source of the magnetic field acts as an actuator for inducing the propeller's rotation. Preferably, the source of the magnetic field is outside the medium which at least partly, preferably completely, surrounds the propeller.

The magnetic field preferably is rotated, thereby inducing a rotation in the propeller. As the magnetic moment of the propeller tends to align with the external magnetic field and the propeller rotates along the axis that exhibits minimal resistance, the orientation of the propeller is determined by the rotating external magnetic field. The magnetic field can be applied foe example by a set of electric coils, e.g. Helmholtz coils, or permanent magnets.

Preferably, if the magnetic field exerts a magnetic gradient force on the propeller in the direction of locomotion, this force is so weak that alone it cannot effect locomotion of the propeller. More preferably, the magnetic field has no gradient component in the direction of locomotion. The preferred magnetic field is stronger than 1 G (gauss), more preferably stronger than 10 G, more preferably stronger than 50 G. The preferred magnetic field is weaker than 10 000 G, more preferably weaker than 1 000 G, more preferably weaker than 500 G, for example 100 G.

Preferably, for inducing the rotation by means of a magnetic field, the propeller is at least partly magnetized or a component materially connected with the propeller is at least partly magnetized. The magnetization is preferably permanent. For this purpose, the propeller comprises a magnetised or magnetisable material; for example, it consists of the magnetised or magnetisable material, or it contains magnetised or magnetisable material, or it is coated with the magnetised or magnetisable material. Suitable materials include Fe, Co, Ni, or magnetic alloy, preferably comprising some or all the afore-mentioned metals. The preferred magnetisable material of the propeller is magnetized in the direction of the maximal length on its cross-section.

In addition or alternatively, an actuator is provided that, like the propeller, is at least partly, preferably completely, surrounded by the medium and materially connected with the propeller. For example, the actuator may be an electrical or a molecular motor; the material connection may comprise a drive shaft. An energy reservoir—such as an electrical battery—for this actuator may likewise be at least partly, preferably completely, surrounded by the medium; preferably, in this case a material connection—for example a wire or a tube—is provided between the reservoir and the actuator to provide the actuator with the energy source, for example electricity or a chemical stored in the energy reservoir. In addition or alternatively the actuator preferably is provided with an energy receiver to receive energy in an untethered fashion from an energy transmitter located the space outside the medium, ie there is no material connection between the energy transmitter and the energy receiver.

Preferably, the torque applied to the propeller when inducing the rotation of the propeller(s) is smaller than 100 mN·mm (millinewton millimetres), preferably smaller than 50 mN·mm, 10 mN·mm, 5 mN·mm, 1 mN·mm.

Preferably the propeller is operated at a speed below 0.9 times its step-out frequency, more preferably below 0.8 times, more preferably below 0.7 times, more preferably below 0.5 times the propellers step-out frequency. Preferably the propeller is operated at a speed above 0.05 times its step-out frequency, more preferably above 0.1 times, more preferably above 0.2 times, more preferably above 0.3 times the propellers step-out frequency. In the context of the present invention, the “step-out-frequency” is the frequency at which the torque is not strong enough to overcome the medium's drag forces. The step-out frequency can for example be measured by driving the propeller with a rotating magnetic field; If the magnetic field rotates sufficiently slowly, the propeller synchronously rotates with the field. There exists a field rotation frequency, however, above which the applied magnetic torque is not strong enough to keep the propeller synchronized with the filed. This is the step-out-frequency.

In a preferred method according to the invention the propeller is completely surrounded by the medium. With this embodiment of the invention particularly strong propulsion can be achieved as all parts of the propeller are in permanent contact with the medium.

The preferred medium is viscoelastic. In the context of the present invention “viscoelastic” refers to media which exhibit both viscous and elastic characteristics when undergoing deformation. A particularly preferred medium is a viscoelastic fluid, where the viscous property is dominant over the elastic property at the applied shear frequency (or shear stress), for example synovial fluid, vitreous humour, mucus. Another particularly preferred medium is a viscoelastic solid, where the elastic property is dominant over the viscous property at the applied shear frequency (or shear stress), for example connective tissue, brain tissue, Matrigel®.

The preferred medium is a biological tissue. A particularly preferred biological tissue is tissue of the brain, the kidney, the prostate, the urinary bladder, a blood vessel, the liver, the pancreas, the breast, the lung, the skin, fat tissues, connective tissues, vitreous humour, mucus, or tumour tissue.

Preferably, the rotation of the propeller induces a strain in the medium, whereby the strain causes a change in the medium's elastic energy, which in turn causes the translation of said propeller.

In a preferred embodiment of the invention a load is attached to the propeller for being moved relative to the medium by the propeller. The preferred load may comprise molecules, nanoparticles, porous polymer matrix, porous silicon and/or one or more electric circuits that are attached to the propeller. Advantageously, the electronic circuit(s) may control the motion of the propeller. Alternatively or in addition one or more tubes and/or wires which are pulled from outside to inside of the medium may be attached to the propeller.

The trajectory of the locomotion preferably is controlled remotely, for example by changing the direction and/or rotational frequency of the magnetic field or by changing the direction, rotational axis, direction of rotation and/or rotational frequency of the actuator at least partly surrounded by the medium. Also, multiple propellers according to the invention may be combined into one device, and in such case individually changing the rotational frequency of the propellers can be used control the propulsion direction of the device. Preferably a controller, for example an appropriately equipped and programmed PC is connected with the actuator (eg the source of the magnetic field or the actuator at least partly surrounded by the medium) to control the trajectory of the locomotion.

The trajectory of the locomotion preferably is imaged and/or measured, for example by one or more or the following imaging methods: light microscopy, fluorescence imaging, x-ray imaging, computer tomography (CT), magnetic resonance imaging (MRI), positron emission tomography (PET), infrared imaging, ultrasound imaging.

The propeller may for example be made of or comprise one or more metal, for example copper, gold, cobalt, nickel, iron, steel, titanium, and or one or more polymer, for example Teflon, PLA, PMMA, PC, and or one or more semiconductor, for example silicon, or a combination of such materials. In a particularly preferred embodiment of the invention, the propeller can be made of biodegradable material. It is achievable advantage of this embodiment of the invention that after deployment into the tissue, no retrieval is needed as it can be degrade and absorb by the body. The propeller may for example consist of two or more sections, one rigid section for propulsion and one biocompatible section for drug carrying and release.

Suitable methods of manufacturing the propeller include moulding, in particular injection moulding, electrodeposition, direct writing, 3D printing and machining. A preferred manufacturing method comprises the steps of (1) defining a straight helical axis; (2) providing a plate extending along the helical axis, the aspect ratio of at least one cross section—preferably all cross sections—of the plate, which cross section(s) is/are related to the helical axis, is/are 2 or more; and (3) applying to the plate a torque along the helical axis, thereby twisting the plate into helical shape. In a next step, the helix can be cut into one or multiple individual propeller(s) of the desired length. It is an achievable advantage of this method that the propeller can be manufactured easy and reliably.

Another particularly preferred manufacturing method comprises the steps of (1) providing a first structure with a defined geometry; (2) moulding of the first structure in a second material, removing the first structure from the second material to generate a negative replicate of the first structure; (3) injecting a moulding material into the negative mould and curing the moulding material to form a second solid structure under given physical and chemical conditions; and (4) releasing the second solid structure from the negative mould, thereby obtaining the defined propeller, wherein the moulding material is a mixture of at least two component materials. Preferred component materials include polymer materials, magnetic materials, drug molecules, radioactive materials. Thus the moulding material may for example consist of a polymer material and a magnetic material.

The curing conditions preferably include at least one of the following: temperature, pH, magnetic field, electric field, acoustic field, light field and radiation. For example, the mixture is epoxy resin mixed with ferromagnetic particles, and the polymer is cured at room temperature within a magnetic field in the direction perpendicular to the helical axis;

Drugs can be incorporated in mixture in the step (3) above, or be absorbed to the propeller materials after releasing in the step (4) above. With this method, the structure, magnetization and functionalization of the propeller can be achieved in a single process.

### BRIEF DESCRIPTION OF THE FIGURES

The invention is illustrated in greater detail with the aid of schematic drawings: Additional FIG. 1(a) is a perspective view of an embodiment of the propeller according to the Invention in perspective view;

Additional FIG. 1(b) is a cross sectional view of the propeller of FIG. 1(a)

Additional FIG. 2 is a light microscope image of a propeller according to the invention to which a magnet is attached and which is embedded in soft tissue;

Additional FIG. 3 shows two light microscope images of the propeller of FIG. 2 penetrating a Matrigel®, the bottom image taken 18 seconds after the top image;

Additional FIGS. 4(a) to (d) schematically compare the cross-sectional shapes of a propeller according to the invention as shown in FIG. 4(a) with those of prior art propellers as shown in FIGS. 4(b) to 4(d);

Additional FIG. 5 is a schematic cross-sectional representation of a propeller according to the invention with medium co-rotating with the propeller;

Additional FIG. 6(a) shows a frame from a video of the propeller in a viscoelastic medium with tracer particles embedded in the medium to visualize the deformation of the medium;

Additional FIG. 6(b) indicates the trajectory of one tracer particle over the period of many rotations of the propeller; the large normalized deformation provides large axial propulsion force;

Additional FIG. 7 illustrates in a cross-sectional view a propeller according to the invention rotating in a viscoelastic medium and the effectively deformed area of the medium induced by the rotation of the propeller is labelled with hatch;

Additional FIG. 8 illustrates in a cross-sectional view a prior art propeller design rotating in a viscoelastic medium and the effectively deformed area of the medium induced by the rotation of the propeller is labelled with hatch;

Additional FIG. 9 illustrates in a cross-sectional view another prior art propeller design rotating in a viscoelastic medium and the effectively deformed area of the medium induced by the rotation of the propeller is labelled with hatch;

Additional FIG. 10 is a force diagram of a short part on the edge of a propeller according to the invention;

Additional FIG. 11 shows two light microscope images of the propeller of FIG. 2 penetrating porcine brain tissue, the bottom image taken 300 seconds after the top image;

Additional FIG. 12 illustrates the method of producing a propeller according to the invention;

Additional FIG. 13 illustrates another method of producing a propeller according to the invention; and

Additional FIG. 14 is a perspective view of two propellers according to the invention with forward tapers at both ends.

Below, these additional figures are referred to as “Figures”.

### DETAILED DESCRIPTION OF THE INVENTION

Propeller Moving in a Tissue Model

It is an achievable advantage of the propeller 1 according to the invention that it can efficiently self-propel through a viscoelastic medium, for example a biological tissue. In FIG. 2, a propeller 1 according to the invention is shown that is fully embedded in a gel medium 2 of Matrigel®, a hydrogel that is used as a tissue model for the validation of the propeller. Matrigel®, available from Gibco®, Life Technologies® is the trade name for a gelatinous protein mixture secreted by mouse sarcoma cells. It resembles the complex extracellular matrix (ECM) found in many tissues, and it is widely accepted as an in vitro model for cell 3D culture, tumour cell metastasis studies and cancer drug screening. Here, Matrigel® serves as a gel medium 2 model for connective tissues for the propeller 1 to penetrate. The Matrigel® solution was used as received, thawed on ice and gelled in an incubator under 37° C. for 1 hour.

The propeller 1 was inserted into the gel medium 2 by means of tweezers. A magnetic field with a homogeneous magnitude (adjustable from 50 to 1000 Gauss) and a continuous rotating direction (frequency in the range of 1 to 100 Hz (hertz) was applied, and the field was rotated with a speed of 10 Hz. One end of the propeller 1 a cylindrical magnet 3 of a neodymium, iron and boron (NdFeB) material, 200 μm (micrometres) in diameter and 400 μm in length and magnetized in the diameter direction is attached in a torque-proof fashion. The magnet has a permanent magnetic moment and rotates together with the external rotating magnetic field. Due to the special shape design of the propeller, it couples the rotation to translational motion (forward or backward propulsion) and achieves net displacement in the gel medium 2 or biological tissues.

As can be best seen in FIG. 1(a), the propeller 1 has a chiral, more precisely a helical shape. It is left-handed but of course a right-handed design would be suitable likewise. The axis of rotation 4 and the helical axis coincide in the propeller of FIG. 1. The direction of locomotion v is indicated as a rightwards arrow v. The direction of rotation is indicated as a semi-circular arrow w. As can be seen in the cross-sectional view in FIG. 1(b), the aspect ratio of any cross-section 5 of the propeller 1 perpendicularly to the helical axis is considerably larger than 5. The aspect ratio is obtained by dividing the largest radius 6 of the cross section by the smallest radius 7 of the cross section 5. The radii 6, 7 extend from the point 8 where the rotational axis 4 perpendicularly pierces the cross section 5 to a point of the circumference 9 of the cross section.

From the images in FIG. 3 it can be seen how the propeller 1 propagates through the Matrigel® gel medium 2. The bottom image was taken 18 seconds after the top image. The dotted line indicates the initial position of the magnet 3. A speed of approximately 45 μm/s (micrometres per second) along the helical axis of the propeller was observed at the rotational frequency of 10 Hz. By choosing the rotational direction of clockwise or counter-clockwise, the propeller 1 can move either forward or backward.

In FIGS. 4(a) to (d) schematically the cross-sectional shape of a propeller 1 according to the invention is compared with cross-sectional shapes of propellers known from the afore-mentioned publications by L Zhang, J J Abbott, L X Dong, B E Kratochvil, D Bell, and B J Nelson, FIG. 4(b), A Ghosh and P Fischer, FIG. 4(c) and T Qiu, J Gibbs, D Schamel, A Mark, U Choudhury, and P Fischer. FIG. 4(d). In the top row, 3D views are provided while in the bottom row the cross-sectional shapes are shown. It can be seen that the cross section 5 of the propeller of the present invention has a considerably larger aspect ratio than the cross sections 5′ of the prior art propellers 1′ based on their radii 6′ and 7′.

Moreover, as the propeller 1 rotates in and moves through the viscoelastic medium 2, parts 10 the medium 2 may attach to the surface of the propeller 1 and rotate together with it. This is schematically shown in FIG. 5. In the example of FIG. 5 the aspect ratio of the cross section of the rotating body that comprises the propeller 1 and the parts 10 of the medium 2 that rotate with the propeller 1 is still larger than 3. The aspect ratio in this case is obtained by dividing the largest radius 11 of the cross section of the rotating body by the smallest radius 12 of the cross section of the rotating body. The radii 11, 12 extend from the point 8 where the rotational axis 4 perpendicularly pierces the cross section to a point of the circumference 9 of the cross section of the rotating body. Propulsion mechanism of the propeller

The inventor[s] believe, without prejudice, that the propeller 1 according to the invention when used in viscoelastic media exploits a new propulsion mechanism, which is different from the mechanism for propulsion in viscous fluids as has been published before. FIGS. 6(a) and 6(b) show results of a Particle Imaging Velocimetry (PIV) experiment. In the experiment, fluorescent polystyrene beads (FluoSpheres®, Life Technologies), 15 μm in diameter, were used as tracer particles and mixed in the Matrigel® gel medium 2 to show the movement, in particular the deformation, of the gel medium 2. The beam of a green laser with a wavelength of 532 nm (nanometres) was expanded by a cylindrical lens to a laser sheet and directed on a thin sheet of the Matrigel® gel medium 2. The motion of the propeller 1 and the tracer particles was recorded by a microscope with a long pass filter (0D4-550 nm, Edmund Optics) and a video camera. The position of the tracer particles were analysed by a customized script in Matlab (R2014b, Mathworks), and circled in every frame of the video. The circles can be seen in both FIG. 6(a) and the enlarged FIG. 6(b). In FIG. 6(b) the trajectory of one tracer particle 13 is indicated. The particle 13 follows a closed, essentially elliptical trajectory 14 over a period of many rotations of the propeller 1. A normalised deformation can be calculated as the quotient of radial displacement d and the distance r from the rotational axis.

The experiment suggests that the movement (deformation) of the viscoelastic medium 2 is clearly different from the flow around a propeller in a viscous fluid. In a fluid, the particles rotate together with the propeller for a full rotation, and the difference of fluidic dynamic drag in the two perpendicular directions at low Reynolds number results in a forward propulsion force, which was explained in the literature. However, a different motion trajectory of the particles was observed with the propeller 1 disclosed here, suggesting that the new design of the propeller 1 enables a new propulsion mechanism in the viscoelastic media, which has not been reported before.

The relaxation time of the viscoelastic solids, which include most biological tissues, are often on the order of minutes, whereas the propeller typically rotates at a frequency of 1 to 10 Hz. As, accordingly, the cycle time (0.1 to 1 s) of the propeller's 1 rotation is much shorter than the relaxation time, only the elastic response of the gel needs to be considered. As an example, shown in the FIG. 7, the cross-section 5 of the propeller 1 is modelled as a rectangular solid that rotates in an initially rectangular hole 15 of the medium 2. Note that in FIG. 7(b) the medium 2 is not flowing but is deformed as the propeller 1 rotates. Large deformation (strain) of the medium 2 is induced by the rotation of the propeller 1. The effectively deformed volume of the medium 2 around the propeller 1 is dramatically larger than in prior art propeller designs (as shown exemplarily in FIG. 8 where the corresponding elements are a propeller 1′ in the form of a screw reported in prior art; and FIG. 9 where the corresponding elements are a propeller 1′ in the form of a conventional screw. The medium 2′, the gap 15′ and the effective deformed area in hatch are also shown in the figures). The medium 2 is considered elastic, ie a spring where the recoil force is positively correlated to the deformation. Therefore, larger deformation of the medium 2 requires more torque for rotation, and exerts larger forward propulsion force. Both of these two phenomena were observed in the experiment.

For further illustration, in FIG. 10 the force diagram of a small section of the propeller 1 (left-handed, the front edge of the propeller rotates upwards in order to move to the right) is shown. The direction of rotation is indicated as an upwards arrow v. It is clear from the force diagram that there is a propelling force component F_p pointing towards the right. Similarly to the situation shown in FIG. 7, the larger the deformation, the larger is the forward propulsion force. Therefore, the proposed propulsion mechanism of the propeller 1 according to the present invention can be summarized in the following three aspects: First, the rotation of the propeller 1 induces large deformation of the gel medium 2. More specifically, large aspect ratio on the cross section 5 of the propeller 1 induces large deformation of the gel medium 2, which leads to large forward propulsion force F_p. Second, the pressure on the tip 16 of the propeller 1 should be higher than the tensile strength of the gel medium 2 in order to break it. It requires an area of the tip 16 as small as possible, for example, a sharp tip 16 is preferable. Moreover, the newly cut area (crack) 15 of the medium 2 due to the forward motion of the tip 16 of the propeller also has a high aspect ratio, such as a rectangular shape, shown as the white area in FIG. 7(a), which again allows the large deformation of the medium 12 when the propeller 1 rotates. It is different from the traditional propeller's 1′ design that the crack 15′ is almost circular, see FIG. 9(a), and the deformation of the medium 2′ induced by the traditional propeller 1′ is small. Third, after the possible attachment of the medium 2 around the propeller 1 such as FIG. 5, it should still fulfil the two conditions above. This criterion ensures a continuous movement of the propeller 1 in the tissue.

The traditional propeller 1′ designs with a hollow opening in the middle, such as the published designs shown in FIG. 4(b) and FIG. 4(c), do not propel efficiently in viscoelastic media. The reason lies in that the opening is filled with the viscoelastic medium during rotation of the propeller, and when considering the medium rotating together with the propeller, the overall structure does not have a high aspect ratio on any cross-section, as shown in FIG. 8(b). In other words, a plug of the gel changes the traditional propeller shape into an almost cylindrical shape, inducing very limited deformation of the media around it, thus the traditional propellers can only rotate at the same position in the viscoelastic medium and no net displacement can be achieved. The present invention in a preferred embodiment clearly differs from the prior art designs in that on at least one cross section, preferably all the cross sections, which are perpendicular to the helical axis of the propeller, the axis passes through the propeller. Or in other words, on at least one cross-section, preferably all the cross-sections, the rotational centre is inside of the propeller.

For some particular kinds of viscoelastic media, such as a yield-stress fluid, the propeller can break (or liquefy) part of the medium due to the shear stress induced by the rotation of the propeller. And the transportation of the broken (or liquefied) parts of the medium to the backwards can also result in the forward propulsion of the propeller.

Preferably, the rotational speed that leads to highest propulsion speed should be used to actuate the propeller 1. This value, which depends on both the geometry of the propeller 1 and the rheology of the medium, can be determined experimentally by sweeping the frequency and measuring the propulsion speed. It has been found that the optimal frequency in a viscoelastic medium 2 of the propeller 1 disclosed here can be much lower than the step-out frequency. When the frequency is increased above the optimal value, the propeller 1 continues to rotate, but the propulsion speed dramatically decreases until it reaches zero. On the contrary, in viscous fluids at low Reynolds number, the optimal frequency of a propeller 1 is very close to the step-out frequency, and the propulsion speed increases linearly with the driving frequency before it reaches step-out. This observation too, suggests that the present propeller 1 enables a new propulsion mechanism in viscoelastic media.

Propeller Moving in a Brain Sample

The light microscope photos in FIG. 11 show a propeller 1 according to the invention that penetrates a porcine brain tissue to demonstrate its capability to move through real biological soft tissues. Fresh porcine brain was stored on ice and received from a local slaughterhouse. A volume of about 25×25×8 mm³ (cubic millimetres) of the brain was dissected, and the propeller 1 was inserted by tweezers. As the tissue was relatively thin, and bright white light back illumination was used, the movement of the propeller was observed inside the brain tissue. The dotted line indicates the initial position of the propeller 1. An average propulsion speed of approximately 35 μm/s was measured at a rotational frequency of about 1 Hz. Due to the shape of the propeller 1, the rotation of propeller 1 can be actuated with limited magnetic torque. In the experiment, a magnetic field with a magnitude of 100 to 300 G was sufficient to drive the propeller 1 through the brain tissue sample. This field is applicable with common magnetic field generators, such as electric coils or permanent magnets setup as discussed in more detail further below.

Fabrication of the Propeller

A method of producing the propeller 1 according to the invention is illustrated in FIG. 12. The propeller 1 was made of copper with a mechanical machining approach. A copper wire, 50 μm in diameter, was mechanically rolled into a flat plate 17 with a width of 255 μm and a thickness of 13 μm. As shown in FIG. 12, the plate 17 was mounted between two concentric clamps 18, 19 which can be rotated relative to each other. By rotating one 18 of the clamps while leaving the other 19 stationary, the plate 17 was twisted into a chiral structure. During twisting, a normal force occurs on the axial direction v, thus the distance between the two clamps 18, 19 was adjusted accordingly. Sensors can be used to measure the force and torque during this process, and the distance and angular position of the clamp can be controlled by motors with a computer. The pitch dimension and chirality of the propeller can be controlled in this way. The long twisted plate 17 was subsequently cut into individual propellers 1 with a desired length of 2 mm. Finally, a miniaturized magnet, 200 μm in diameter and 400 μm in length, was attached to one tip of the propeller 1.

The cutting procedure can be done by machining, laser etching, (focused) ion etching, or chemical etching. The mask for etching can be fabricated by photolithography on the two sides of the plate before the twisting process. In this way, a mass production process of the propeller can be achieved.

Another method of producing the propeller 1 according to the invention is illustrated in FIG. 13. A structure of the propeller 1 is first obtained, for example in copper material by the method described above or by 3D printing (FIG. 13(a)); then, the structure is moulded into a second material, such as a soft polymer, eg PDMS (FIG. 13(b)); the first structure is removed from the negative mould 20, for example by rotating the propeller 1 in the right direction and it propels out of the mould 20, or by expanding the soft polymer mould 20 (FIG. 13(c)); liquid polymer material or mixture is injected into the negative mould 20 (FIG. 13(d)), for example a mixture of epoxy resin and ferromagnetic particles (mean diameter 40 μm), the polymer is cured at room temperature in the presence of an external magnetic field as illustrated by the arrow in the FIG. 13(d); finally, the propeller 1 is obtained by releasing it from the negative mould 20, either by breaking the mould 20, or by rotating the propeller 1 in the right direction and it propels out of the mould 20 (FIG. 13(e)). The propeller 1 have the right magnetic moment M (as indicated by arrow in (FIG. 13(f)), as the magnetic particles in the structure are aligned in the right direction when the external magnetic field B is applied. Drugs can be incorporated in polymer mixture in the moulding step above, or be absorbed to the propeller materials after releasing in the last step above.

FIG. 14 illustrates how the two tips 16 of the propeller 1 can be cut or etched or moulded into designed shape, preferably a sharp tip. This way, the pressure at the tip 16 can be increased by decreasing the contact area; also, the shear rate in the medium 2 in front of the propeller can be increased. As many biological media are shear-thinning, a larger shear rate also helps the forward propulsion of the propeller. In this case, the sharp tip of the propeller is preferably at the edge of the propeller tip 16 and far from the rotational axis.

Actuation of the Propeller

A suitable setup for inducing rotation into the propeller by means of a rotating magnetic field is for example known from the afore-mentioned publication by T Qiu, J Gibbs, D Schemel, A Mark, U Choudhury, and P Fischer. The relevant parts of this document are incorporated into the present disclosure by reference.

The field can be spatially homogeneous or with a magnetic gradient in space, but preferably the pulling force acting on the propeller generated by the magnetic gradient is in the same direction as the direction of the self-propelling force of the propeller 1. The magnetic field can be generated with electric coils. For example, three pairs of Helmholtz coil can achieve the motion control of the propeller in three dimensional space by changing the phase and magnitude of the current in different coils. The magnetic field can also be generated with the rotation of permanent magnet(s), which can be several magnets specially arranged in space or only one magnet keeping a required distance away from the propeller. To control the propulsion trajectory with the permanent magnets setup, the rotational axis of the setup should be changed.

For the realisation of the invention in its various embodiments, the features disclosed in the present description, claims and drawings can be of relevance individually as well as in any combination.

1. A method which facilitates the diffusion or active transport of a particle through a medium, wherein in order to avoid adhesion of the particle to the medium, the particle is coated with at least one solid layer linked to the surface of the particle and/or at least one liquid layer that surrounds the solid layer.

2. A particle, wherein in order to avoid adhesion of the particle to a medium, the particle is coated with at least one solid layer linked to the surface of the particle and/or at least one liquid layer that surrounds the solid layer.

3. The particle of claim 2, wherein the characteristic size of the particle is equal to or smaller than the mesh size of the medium.

4. The particle of claim 2, wherein the characteristic size of the particle is larger than the mesh size of the medium, preferably not larger than 1 000 times, more preferably not larger than 10 times the mesh size, and/or wherein the solid layer has a thickness of between 0.2 nm and 20 μm and/or the liquid layer has a thickness between 0.5 nm and 500 μm.

5. The particle of claim 2, wherein the coating material of the solid layer or the liquid layer contain one or more components from the group of components consisting of: poly(ethylene oxide), poly(4-styrenesulfonic acid), poly(sodium 4-styrenesulfonate), polyethylene glycol, siloxane, perfluorocarbon, negative charged polyelectrolytes, hyaluronic acid, poloxamer, enzymes, albumin, polysaccharides, poly(vinyl acetate), or poly(vinylpyrrolidone).

6. The method of claim 1, wherein the particle is dispersed in aqueous solution, prior to the application in the medium and/or wherein the particle is directly applied in the medium.

7. The method of claim 6, wherein the particle is dispersed in aqueous solution, prior to the application in the medium, at least one stabilizer is added in the aqueous solution to keep the particles dispersed, and the stabilizer includes at least one component from poly(vinyl alcohol), polyvinylpyrrolidone, poly(ethylene oxide), polyethylene glycol, poly(4-styrenesulfonic acid), poly(sodium 4-styrenesulfonate), hyaluronic acid, poloxamer, starch, dextrin, chitosan, alginate, isolated soy protein, gelatin, catalase, whey protein, albumin, histones, carrageenan, xanthan gum, phenylpropanamide, sodium benzenesulfonate.

8. The particle of claim 2, wherein the particle has therapeutic functions, and/or wherein the particle is used to aid biomedical imaging or diagnostics imaging, and/or wherein particle is radioactive, or generate heat or light radiation under an external stimulus, and/or wherein the particle is associated with, or contacts a therapeutic agent.

9. The particle of claim 2, wherein the particle has a chiral or modified chiral part, and/or wherein the particle has a part in helical shape, and/or wherein the particle has a magnetic moment.

10. The method of claim 1, wherein the particle diffuses through a medium that is biologically relevant, including human or animal vitreous humor, mucus, synovial fluids, lymphatic fluids, cells, connective tissues, the tissues of brain, nerve, heart, lung, kidney, blood vessel, liver, pancreas, gall bladder, GI tract, urinary tract, testicle, penis, female reproductive tract, breast, prostate, ear, nose, appendix, joint and bone, or wherein the particle is transported by the application of an external force or torque through a medium that is biologically relevant, including human or animal vitreous humor, mucus, synovial fluids, lymphatic fluids, cells, connective tissues, the tissues of brain, nerve, heart, lung, kidney, blood vessel, liver, pancreas, gall bladder, GI tract, urinary tract, testicle, penis, female reproductive tract, breast, prostate, ear, nose, appendix, joint and bone.

11. The method of claim 1, wherein the motion of the particle is induced remotely by means of a magnetic field.

12. A method for producing a particle with a coating, wherein the method comprises the steps of:
fabrication of the particle in defined shape;
coating a solid layer that links to the surface of the particles; and
coating a liquid layer that fuses with the said solid layer.

13. A method for utilizing a particle with a coating, wherein the method comprises the steps of:
suspend the particle in an aqueous solution;
injection the suspension into a medium;
apply a magnetic field to induce the movement of the particle; and
observe the movement with an imaging technique.

14. A method for utilizing a particle with a coating, wherein the method comprises the steps of:
disperse the particle into a medium;
apply a magnetic field to induce the movement of the particle; and
observe the movement with an imaging technique.

15. The method of claim 13, wherein the magnetic field is altered based on the feedback of the imaging results, and the particles are guided to a target location in the said medium.
